(19) United States
(12) Patent Application Publication
(10) Pub. No.: US 2006/0025327 A1
(43) Pub. Date: Feb. 2, 2006

(54) Hybrid molecules QA, wherein Q is an aminoquinoline and a is an antibiotic or a resistance enzyme inhibitor, their synthesis and their uses as antibacterial agent

(75) Inventors: Muriel Sanchez, Balma (FR); Bernard Meunier, Castanet-Tolosan (FR)

Correspondence Address: IP LAW SPECIALISTS, LLC, 7777 LEESBURG PIKE, SUITE 304 NORTH, FALLS CHURCH, VA, 22043, US

(73) Assignee: Palumed S. A. & Centre National de la Recherche Scientifique CNRS

(21) Appl. No.: 11/019,450

(22) Filed: Dec. 23, 2004

(30) Foreign Application Priority Data
Jul. 30, 2004 (FR) FR 0408441

(51) Int. Cl. A61K 38/17; C07K 14/47

(52) U.S. Cl. 514/2; 530/300; 514/200; 540/222

(57) ABSTRACT

Aminoquinoline-antibiotic hybrid compounds in the form of hybrid molecules QA, wherein Q is an aminoquinoline and A is an antibiotic or a resistance enzyme inhibitor, their synthesis and their uses as antibacterial agent. This compound is defined by the general formula (I):
Q-(Y₁)ₚ—(U)_{p′}—(Y₂)_{p″}-A (I) in which
Q represents an aminoquinoline, (Y₁)ₚ—(U)_{p′}—(Y₂)_{p″}— is an optional spacer arm and A is an antibiotic, one of its derivatives or precursors, or a resistance enzyme inhibitor. The invention unexpectedly enables the activity of the antibiotic to be improved.

### BACKGROUND OF THE INVENTION

An object of the invention is hybrid molecules <<QA>> containing an aminoquinoline moiety (O) which is covalently linked to a residue (A) which is an antibiotic, one of its derivatives or precursors, or a resistance enzyme inhibitor. The invention also relates to their synthesis and their uses as antibacterial agent.

Over the last 50 years, the introduction of penicillin followed by many other antimicrobial agents has represented one of the greatest successes in modern medicine in the treatment of bacterial infections (Greenwood, D. et al. in Antimicrobial Chemotherapy; Greenwood, D., Ed.; Oxford University Press: New York, United States, 2000). The appearance and the propagation of bacterial strains which are resistant to practically all the antimicrobial agents currently available become a serious problem for public health (World Health Organisation. Resistance aux antimicrobiens: une menace pour le monde. Médicaments essentiels: Le Point, 2000, 28 and 29, 1-35. Accessible on www.who.int).

The problem of bacterial resistance is also analyzed by Coates, A.; et al. in Nature Rev. Drug Discov. 2002, 1, 895-910, entitled: <<The Future Challenges Facing the Development of New Antimicrobial Drugs >>.

The aminoquinolines (Q) are known molecules.

Moreover, it has been demonstrated by Malléa in the literature that the aminoquinolines (Q), as a mixture with various classes of antibiotics, inhibited the active efflux of the antibiotics (vide Malléa, M.; et al Alkylaminoquinolines inhibit the bacterial antibiotic efflux pump in multidrug-resistant clinical isolates. Biochem. J. 2003, 376,801-805).

### AIMS OF THE INVENTION

A main aim of the present invention is to solve the novel technical problem which consists of providing a solution which enable finding novel antibiotic molecules less prone to resistance of bacteria.

Another main aim of the invention is to find novel antibiotic molecules more effective than current antibiotics.

Another main aim of the present invention is to solve these novel technical problems by providing novel antibiotic molecules, the manufacture of which is relatively easy according to an inexpensive method which gives good industrial yields.

The present invention solves, for the first time, the whole of these technical problems in a satisfactory, safe and reliable manner, which can be used on an industrial scale, notably on a pharmaceutical scale.

### SUMMARY OF THE INVENTION

The innovative character of the present invention bears upon the preparation and the evaluation of the hybrid molecules <<QA>>. According to the invention, the aminoquinoline part (O) of these novel molecules has been covalently fixed to an antibiotic residue (A), one of its derivatives or precursors, or a resistance enzyme inhibitor.

These hybrid molecules QA are generally named “antibioquines” or particularly “peniciquines”, “cephaloquines” or “quinoloquines” when the A moiety is an antibiotic, a penicillin moiety, a cephalosporin moiety or a quinolone moiety, respectively.

According to the invention, it has been discovered in an unexpected and non-obvious way that the covalent anchoring of an aminoquinoline onto an antibiotic did not lead to a loss of the antibiotic activity, but on the contrary, led to a synergistic effect increasing the antibiotic activity, and this constitutes the basis of the present invention.

Another particularly unexpected effect of the invention resides in the fact that it has been surprisingly discovered that the antibiotic activity was preserved in the case of a covalent bond with an aminoquinoline for various classes of antibiotics. Thus, this unexpected improvement of the activity is not limited to a particular type of antibiotic.

This constitutes a particularly significant technical improvement of the invention insofar as the actual tendency for an antibiotic treatment is no longer the use of broad spectrum antibiotics. Actually, in fact, broad spectrum antibiotics strongly participate in the selection of resistant organisms, and, moreover, they bear within them an inherent danger of deep modifications of the flora with a development of secondary complications which are sometimes dangerous. Hence, the use of antibiotics should tend to the use of an antibiotic which is as selective as possible on the involved bacteria, for a period of time as short as possible.

By virtue of the fact that the invention is not limited to a particular class of antibiotics, it will in contrast thus be possible to modify the various families of antibiotics without reducing their effectiveness.

Another particularly unexpected effect of the invention resides in the fact that it has been surprisingly discovered that the inhibitory activity of inhibitors of resistance enzymes which inactivate antibiotics, notably the antibiotics mentioned above, was preserved in the case of a covalent bond with an aminoquinoline for various classes of resistance enzymes inhibitors. Thus, this unexpected improvement of the inhibitory activity is not limited to a particular type of resistance enzyme inhibitors.

The invention will therefore enable having a panel of molecules at one's disposal which are active on resistant strains and which will be able to be used as a function of their specific activity.

It will be possible for the person skilled in the art to assess the major significance of the present invention, which covalently links an aminoquinoline type moiety (O) to a residue (A) representing an antibiotic residue, one of its derivatives or precursors, or a resistance enzyme inhibitor, or even one of its salts, hydrates, prodrugs and prodrug salts, and an aminoquinoline molecule Q, linked to each other via a covalent bond which is represented by —(Y¹)ₚ—(U)_{p′}—(U₂)_{p″}—, a covalent bond which can be direct or indirect by the use of a spacer arm.

### DETAILED DESCRIPTION OF THE INVENTION

The invention relates essentially to novel hybrid antibiotic molecules which are represented by the general formula (I):
Q-(Y₁)ₚ—(U)_{p′}, —(Y₂)_{p″}-A (I)
in which
Q represents an aminoquinoline-type molecule;
A represents an antibiotic residue, one of its derivatives or precursors, or a resistance enzyme inhibitor, or even one of its salts, hydrates, prodrugs and prodrug salts;
   which are linked together via a covalent bond which is represented by —(Y₁)ₚ—(U)_{p′}—(Y₂)_{p″}—, a covalent bond which can be direct or indirect by the use of a spacer arm.

The residue A, which is an antibiotic, a derivative or precursor of antibiotics, an inhibitor of resistance enzymes, or even one of their salts, hydrates, prodrugs or a prodrug salt, are covalently linked either directly to the aminoquinoline, or to the spacer arm and can be linked notably to Q, Y₁, U, or Y₂, notably as defined below, in any fixing site, notably by reaction with one of the reactive functions of the compounds A. The present invention also relates to their method of preparation, the various uses, pharmaceutical compositions containing them, as well as to a method of therapeutic treatment. These novel molecules can also be used as an antibacterial agent.

According to a first aspect, the present invention provides a hybrid aminoquinoline-antibiotic compound, characterized in that it has the following general formula (I):
Q-(Y₁)ₚ—(U)_{p′}—(Y₂)_{p″}-A (I)
in which:
Q represents an aminoquinoline of the following formula (II) or (III): 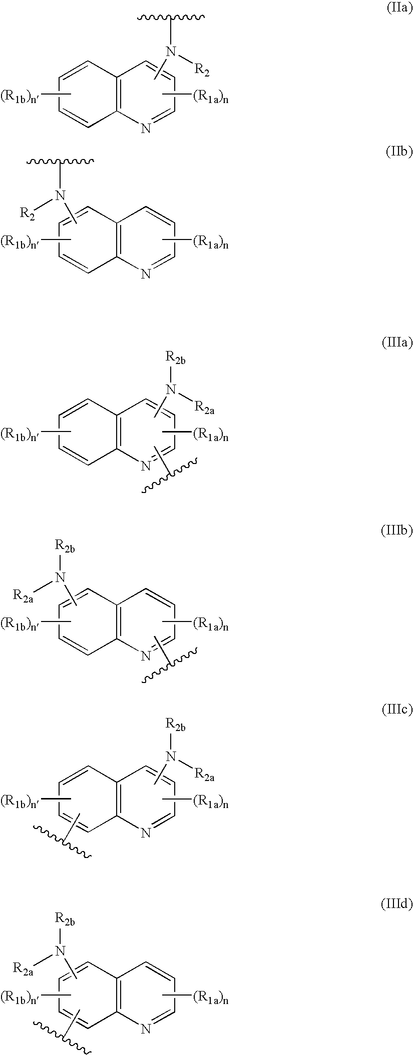
   in the above formulae:
the sign [symbol] indicates the anchoring site of the other fragment, e.g. either Y₁, or U, or Y₂, or A;
n and n′ represent, independently of each other, 0, 1, 2 or 3;
R₁ₐ and R_{1b} represent one or more substituents which are identical or different, occupying any position and representing a moiety which is selected from the group consisting of halogen, hydroxy, trifluoromethyl, trifluoromethoxy, carboxy, amine, sulfate, sulfonate, phosphate, phosphonate, nitro, cyano, aryl, heteroaryl or alkyl, alkylamino, alkoxy, alkylthio, alkylsulfonyl, alkylsulfonamido, alkylsulfonylamino, alkylamido, alkylcarboxy, alkoxycarbonyl, alkylcarbonylamino, said alkyl groups comprising 1 to 6 carbon atoms, which are saturated or unsaturated, linear, branched or cyclic, containing if need be one or more amine, amide, thioamide, sulfonyl, sulfonamide, carboxy, thiocarboxy, carbonyl, thiocarbonyl, hydroxyimine, ether or thioether moieties and themselves being able to bear 1 to 4 substituents, which are identical or different, and which are selected from halogen, hydroxy, trifluoromethyl, trifluoromethoxy, carboxy, carbonyl, amine, nitro, aryl, or heteroaryl,
R₂ₐ and R_{2b} being substituents which are identical or different, being able if need be to form a cyclic structure together or with Y₁, Y₂, U or A and representing a hydrogen atom or a linear, branched or cyclic C1 to C6 alkyl moiety containing if need be one or more amine, amide, thioamide, sulfonyl, sulfonamide, carboxy, thiocarboxy, carbonyl, thiocarbonyl, ether or thioether moieties and being able to bear 1 to 4 substituents, which are identical or different, and which are selected from halogen, hydroxy, trifluoromethyl, trifluoromethoxy, carboxy, carbonyl, amine, nitro, aryl, or heteroaryl,
p, p′, p″ are, independently of each other, 0 or 1,
Y₁ and Y₂, which are identical or different, represent a saturated or unsaturated, linear, branched or cyclic C1 to C6 alkyl chain, containing if need be one or more amine, amide, thioamide, sulfonyl, sulfonamide, carboxy, thiocarboxy, carbonyl, thiocarbonyl, hydroxyimine, ether or thioether moieties and optionally forming a cyclic structure with R₂ including N of the aminoquinoline part Q and/or the U function, the C1 to C6 chain being able to bear 1 to 4 substituents, which are identical or different, and which are selected from the group consisting of halogen, hydroxy, trifluoromethyl, trifluoromethoxy, carboxy, carbonyl, amine, nitro, hydroxyimine, aryl or heteroaryl having 5 to 6 members comprising 1 to 4 heteroatoms selected from nitrogen, sulfur and oxygen and themselves being able to bear one or more substituents selected from the group: halogen, hydroxy, trifluoromethyl, trifluoromethoxy, carboxy, amine, nitro or cyano, the C1 to C6 chain being additionally able to be substituted with 1 to 4 groups of alkyl, alkylamino, alkoxy, alkylthio, alkylsulfonyl, alkylsulfonamido, alkylaminosulfonyl, alkylamido, alkylcarboxy, alkoxycarbonyl, alkylaminocarbonyl, alkoxyimine type, said alkyl groups comprising 1 to 6 carbon atoms which are linear, branched or cyclic themselves being able to contain one or more amine, amide, thioamide, sulfonyl, sulfonamide, carboxy, thiocarboxy, carbonyl, thiocarbonyl, ether, thioether moieties, aryl moieties or heteroaryl moieties having 5 to 6 members comprising 1 to 4 heteroatoms selected from nitrogen, sulfur and oxygen, and themselves being able to bear one or more substituents, which are selected from the group consisting of halogen, hydroxy, trifluoromethyl, trifluoromethoxy, carboxy, carbonyl, amine, nitro and cyano,
U is an amine, amide, thioamide, sulfonyl, sulfonamide, carboxy, thiocarboxy, carbonyl, ether, thioether, thiocarbonyl, sulfonate, alkoxyimine (C═N—OR) or alkoxyiminocarbonyl (C(O)—C═N—OR) function with R representing a hydrogen atom or a C1 to C6 alkyl moiety, which is linear, branched or cyclic, containing if need be one or more amine, amide, thioamide, sulfonyl, sulfonamide, carboxy, thiocarboxy, carbonyl, thiocarbonyl, ether or thioether moieties,
A represents an antibiotic residue, one of its derivatives or precursors, or a resistance enzyme inhibitor, or even one of its salts, hydrates, prodrugs and prodrug salts.

It is understood that the heteroaryl moiety is an aromatic ring having 5 to 6 members comprising 1 to 4 heteroatoms selected from nitrogen, sulfur and oxygen and that the aryl or heteroaryl moieties can themselves bear one or more substituents selected from the group: halogen, hydroxy, trifluoromethyl, trifluoromethoxy, carboxy, amine, nitro or cyano.

In the definition of the compounds of formula (I) above and in the following, the term “halogen” is understood as meaning a fluorine, chlorine, bromine or iodine atom.

According to the preferred compounds of the invention, the Q part of the hybrid molecules of formula (I) represents either an aminoquinoline of formula (II), in which an antibiotic part is linked to the amine function, or an aminoquinoline of formula (III) wherein the antibiotic is directly fixed onto the quinoline nucleus.

According to an embodiment, the hybrid molecules containing an aminoquinoline of formula (II) were prepared from haloquinolines and amine derivatives also containing a reactive function for fixing the antibiotic.

According to another embodiment, the quinoline precursors of the hybrid molecules containing an aminoquinoline of type (III) are aminoquinolines which also possess a reactive function, such as halogen, haloalkyl, hydroxy, amine, sulfonamide or carboxy.

According to the invention which covers compounds of formula (I), A represents an antibiotic residue, one of its derivatives or precursors, or a resistance enzyme inhibitor. This residue can advantageously be selected from the large families of antibiotics which are known to the person skilled in the art, such as, for example, β-lactams, quinolones, oxazolidinones, derivatives of fosfomycin, nitro-imidazoles, nitro-furans, sulfamides, streptogramins, synergistins, lincosamides, tetracyclins, derivatives of chloramphenicol, derivatives of fusidic acid, diaminopyrimidines, aminosides, macrolides, polypeptides, glycopeptides, rifamycins, lipodepsipeptides or amongst the inhibitors of resistance enzymes, such as the inhibitors of β-lactamases. In the following embodiments of compounds of formula (I) covered by the invention, some examples of formulae of the antibiotic A are given as non-limiting examples.

According to an advantageous embodiment of the compounds of formula (I) according to the invention, A can be selected from the family of β-lactams which contains, amongst others: penams (or penicillins) of formula (IV), oxapenams of formula (V), penems of formula (VIa) or (VIb), carbapenems of formula (VIIa) or (VIIb), cephems (or cephalosporins) of formula (VIIIa) or (VIIIb), cephamycins of formula (IXa) or (IXb), oxacephems of formula (Xa) or (Xb), carbacephems of formula (XIa) or (XIb) and monobactams of formula (XII), as follows: 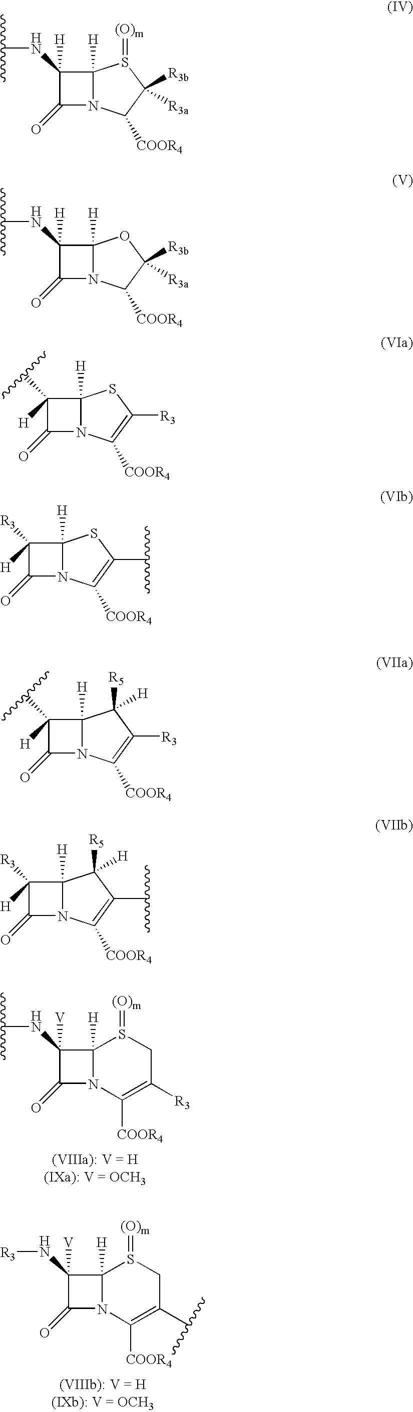 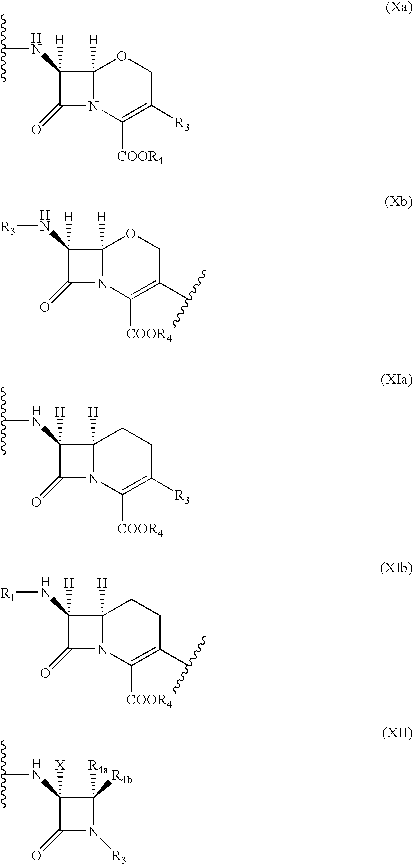
in which
R₃ₐ and R_{3b} represent substituents which are identical or different and which are selected from the group consisting of halogen, hydroxy, trifluoromethyl, trifluoromethoxy, carboxy, amine, sulfate, sulfonate, phosphate, phosphonate, nitro, cyano, aryl, heteroaryl or alkyl, alkylamino, alkoxy, alkylthio, alkylsulfonyl, alkylsulfonamido, alkylsulfonylamino, alkylamido, alkylcarboxy, alkyloxycarbonyl, alkylcarbonylamino, said alkyl groups comprising 1 to 6 carbon atoms, which are saturated or unsaturated, linear, branched or cyclic, containing if need be one or more amine, amide, thioamide, sulfonyl, sulfonamide, carboxy, thiocarboxy, carbonyl, thiocarbonyl, hydroxyimine, ether or thioether moieties and themselves being able to bear 1 to 4 substituents, which are identical or different, and which are selected from halogen, hydroxy, trifluoromethyl, trifluoromethoxy, carboxy, carbonyl, amine, nitro, aryl, or heteroaryl,
R₄ₐ and R_{4b} which are identical or different, being able if need be to form, together, a cyclic structure or a multiple bond, represent a hydrogen atom or a saturated or unsaturated, linear, branched or cyclic C1 to C6 alkyl moiety, containing if need be one or more amine, amide, thioamide, sulfonyl, sulfonamide, carboxy, thiocarboxy, carbonyl, thiocarbonyl, hydroxyimine, ether or thioether moieties and being able to bear 1 to 4 substituents, which are identical or different, and which are selected from halogen, hydroxy, trifluoromethyl, trifluoromethoxy, carboxy, carbonyl, amine, nitro, aryl, or heteroaryl,
R₅ is a hydrogen atom or a saturated or unsaturated, linear, branched or cyclic C1 to C6 alkyl moiety,
V represents a methoxy group or a hydrogen atom.

The β-lactams of formulae (IV), (V), (VIb), (VIIIa), (IXa), (Xa), (XIa) and (XII) were for example coupled to a quinoline moiety by making use of their amine function.

The penems of formula (VIa) and carbapenems of formula (VIIa) were grafted for example onto an aminoquinoline making use for example of a reactive function of hydroxyl type.

The coupling reaction with the carbapenems of formula (VIIb) was carried out for example from a carbonyl or hydroxyl group.

A reactive function of hydroxy, halogen, or alkene type was used advantageously for fixing cephalosporins, cephamycins, oxacephems and carbacephems of respective formulae (VIIIb), (IXb), (Xb) and (XIb).

In another family of compounds according to the invention, A represents a quinolone moiety such as the one described by the following formula (XIII), 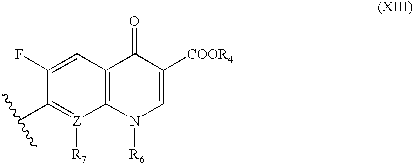
in which
R₄ is as defined above,
R₆ and R₇ are substituents which are identical or different, being able if need be to form, together, a cyclic structure or a multiple bond and representing a hydrogen atom or a substituent which is selected from the group consisting of halogen, heterocycle, aryl or heteroaryl, or an alkyl, alkoxy or alkylamine moiety, said alkyl groups comprising 1 to 6 carbon atoms, which are saturated or unsaturated, linear, branched or cyclic, containing if need be one or more amine, amide, thioamide, sulfonyl, sulfonamide, carboxy, thiocarboxy, carbonyl, thiocarbonyl, ether or thioether moieties and being able to bear 1 to 4 substituents, which are identical or different, and which are selected from halogen, hydroxy, trifluoromethyl, trifluoromethoxy, carboxy, carbonyl, amine, nitro, aryl, or heteroaryl,
Z is a nitrogen or carbon atom.

The term “heterocycle” is understood to mean a 5 to 6 membered ring comprising 1 to 4 heteroatoms selected from nitrogen, sulfur and oxygen, itself being able to bear one or more substituents selected from the group: halogen, hydroxy, trifluoromethyl, trifluoromethoxy, carboxy, amine, nitro or cyano.

A reactive function of amine or halogen type of the quinolones known to the person skilled in the art was advantageously used for the coupling reaction with a quinoline type derivative.

In another embodiment of the compounds according to the invention, A represents an oxazolidinone residue such as those described by the following formulae (XIVa) or (XIVb), 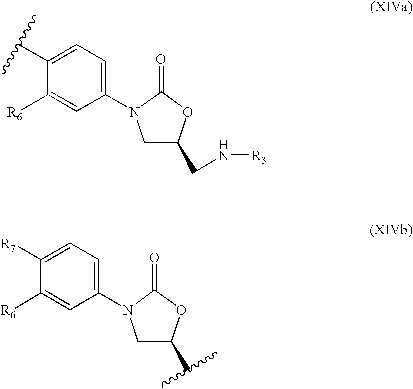
in which R₃, Rr and R₇ are as defined above.

Such hybrid molecules were advantageously prepared either by making use of an amine or halogen type reactive function of an oxazolidinone or by synthesis of the oxazolidinone ring from an aminoquinoline comprising a protected amine function and from <<(R)-glycidyl butyrate>> according to the methods known to the person skilled in the art.

In another embodiment of the compounds according to the invention, A represents a derivative of fosfomycin such as the one described by the formula (XV) as follows, 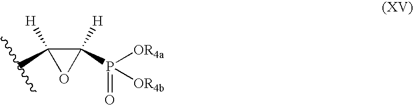
in which R₄ₐ and R_{4b}, which are identical or different, being able if need be to form, together, a cyclic structure are as defined above.

The synthesis of hybrid molecules derived from fosfomycin was for example carried out by epoxidation of an alkene type precursor before or after fixing onto the aminoquinoline.

In another family of compounds according to the invention, A represents a nitro-imidazole residue such as those described by the formulae (XVIa) or (XVIb) or a nitro-furan residue such as the one described by the formula (XVII), as follows, 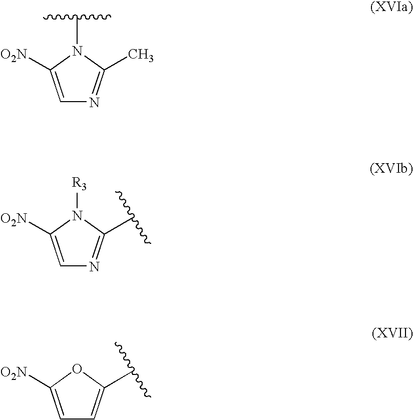
in which R₃ is as defined above.

A reactive function of hydroxy, amine or halogen type was for example used in the coupling reaction of the nitro-imidazole or nitro-furan derivatives of formula (XVI) or (XVII) with a quinoline moiety.

In another embodiment of the compounds according to the invention, A represents a sulfamide residue such as the one described by the following formula (XVIII),

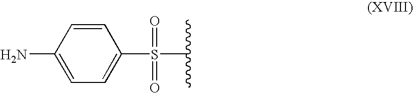

This residue was for example fixed onto an aminoquinoline from a sulfonamide or sulfonic acid type reactive function.

In another family of compounds according to the invention, A represents a streptogramin residue such as those described by the formulae (XIXa) or (XIXb) or a synergistin residue such as the one described by the formula (XX), as follows, 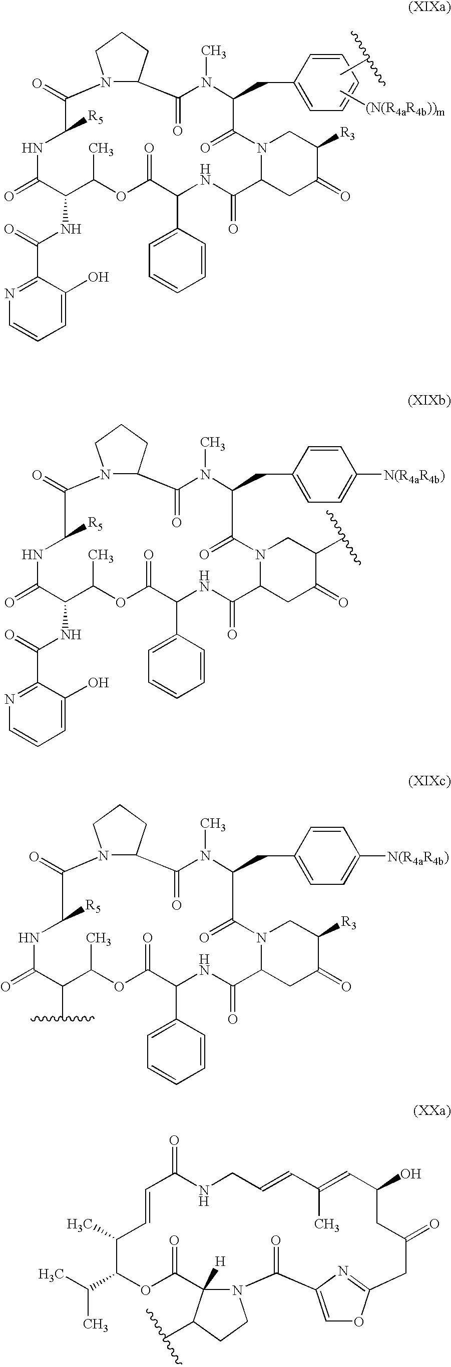 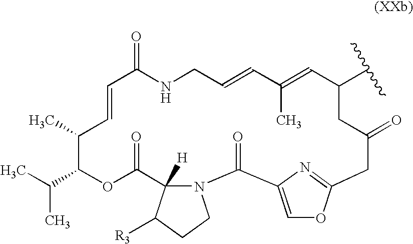
in which R₃, R₄ₐ, R_{4b}, R₅ and m are as defined above.

The synthesis of hybrid molecules incorporating a streptogramin or synergistin derivative was carried out for example from precursors of pristinamycin or virginiamycin type.

In another embodiment of the compounds according to the invention, A represents a lincosamide residue such as the one described by the formula (XXI) as follows,

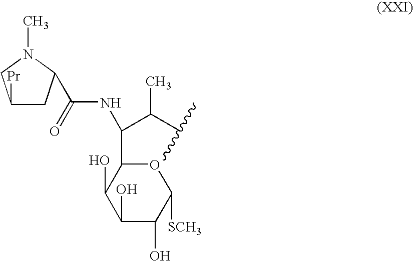

Lincosamides possess a hydroxy function or a halogen atom which was used for example for grafting them onto an aminoquinoline.

In another embodiment of the compounds according to the invention, A represents a tetracyclin residue such as the one described by the formula (XXII) as follows, 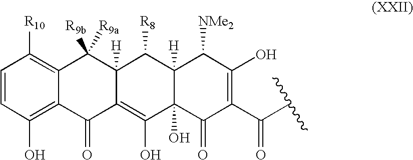
in which
R₈ and R₉ₐ, R_{9b}, which are identical or different, represent a hydrogen atom or a moiety which is selected from the group: hydroxy or methyl,
R₁₀ is a hydrogen atom or a halogen.

The coupling reaction with the tetracyclins of formula (XXII) was carried out for example from their amide function.

In another family of compounds according to the invention, A represents a derivative of chloramphenicol such as those described by the formulae (XXIIIa) or (XXIIIb), as follows, 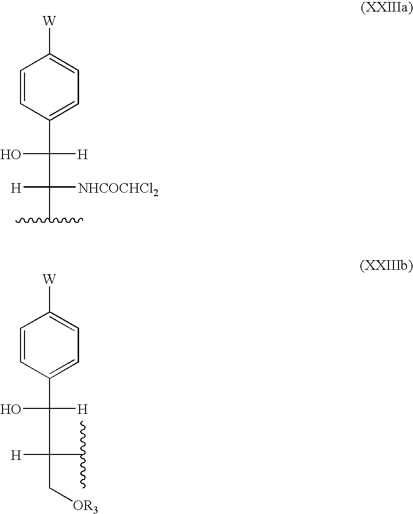
in which
R₃ is as defined above,
W represents an NO₂ or SO₂R₅ moiety, R₅ being as defined above.

A reactive function of hydroxy or halogen type was used for example for fixing the chloramphenicol derivatives according to the modes (XXIIIa) and (XXIIIb). In another embodiment of the compounds according to the invention, A represents a derivative of fusidic acid such as those described by the following formulae (XXIVa), (XXIVb) or (XXIVc), 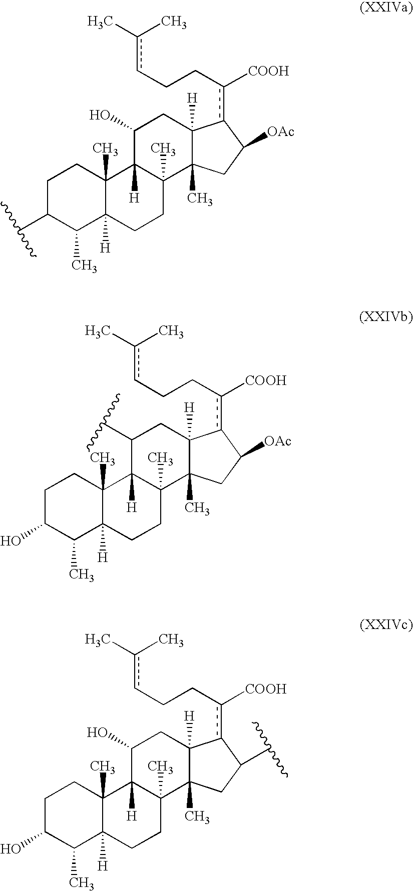
The fusidic acid derivatives of formula (XXIV) as defined above were grafted onto an aminoquinoline for example from a hydroxyl function.

In another family of compounds according to the invention, A represents a diaminopyrimidine residue such as those described by the formula (XXV) as follows, 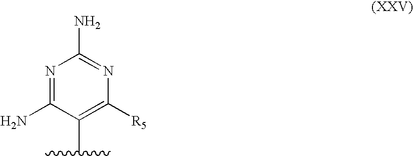
in which R₅ is as defined above.

Hybrid molecules incorporating a diaminopyrimidine residue were prepared notably by making use of a hydroxy or halogen type reactive function of a known diaminopyrimidine.

In another family of compounds according to the invention, A represents an aminoside residue which is formed by the union of a genin moiety from the group of aminocyclitols, with one or more oses at least one of which is an aminosugar, which are linked together via glycosidic bridges. Many aminosides exist with various chemical structures which can be coupled to an aminoquinoline by making use of one of their amino or hydroxy type reactive functions.

In another embodiment of the compounds according to the invention, A represents a macrolide residue having 14, 15 or 16 atoms such as those described the following formulae (XXVI), (XXVII) and (XXVIII), respectively, 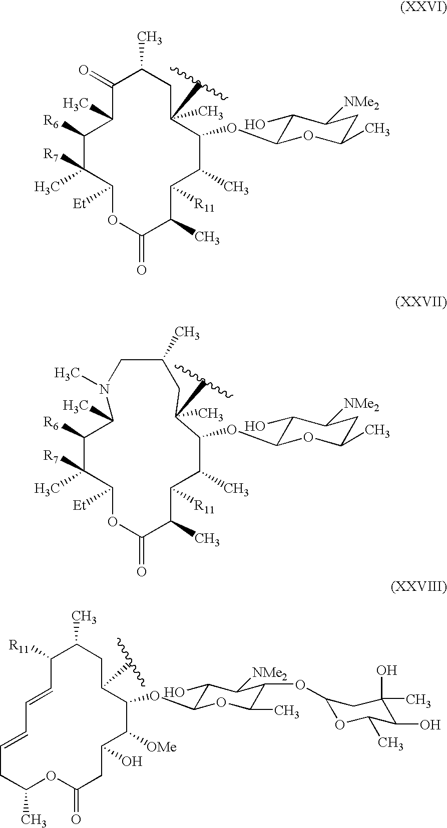
in which
R₆ and R₇ are as defined above,
R₁₁ is an oxygen atom linked via a double bond of carbonyl type to the macrocycle or a hydroxy group or an osidic derivative linked via a glycosidic bridge to the macrocycle and being able to bear 1 to 6 substituents, which are identical or different, and which are selected from hydroxy, alkyl or alkoxy, said alkyl groups comprising 1 to 6 carbon atoms which are linear, branched or cyclic.

In addition to the fixation sites described in the formulae (XXVI) to (XXVIII), other sites can be envisaged for grafting the aminoquinolines onto the macrolides. It is understood that the invention covers the aminoquinoline-macrolide hybrid molecules resulting from these various means of fixation.

Advantageously, the reactive functions of the macrolides of hydroxy, amino or carbonyl type were used for the coupling reaction with the aminoquinolines.

In another family of compounds according to the invention, A represents a polypeptide residue such as derivatives of polymyxines or of bacitracin linking various peptidic structures. These residues were grafted onto an aminoquinoline notably via one of their free amino functions.

In another embodiment of the compounds according to the invention, A represents a glycopeptide residue such as the derivatives of vancomycin described by the formula (XXIX) or the derivatives of teicoplanin described by the formula (XXX), as follows, 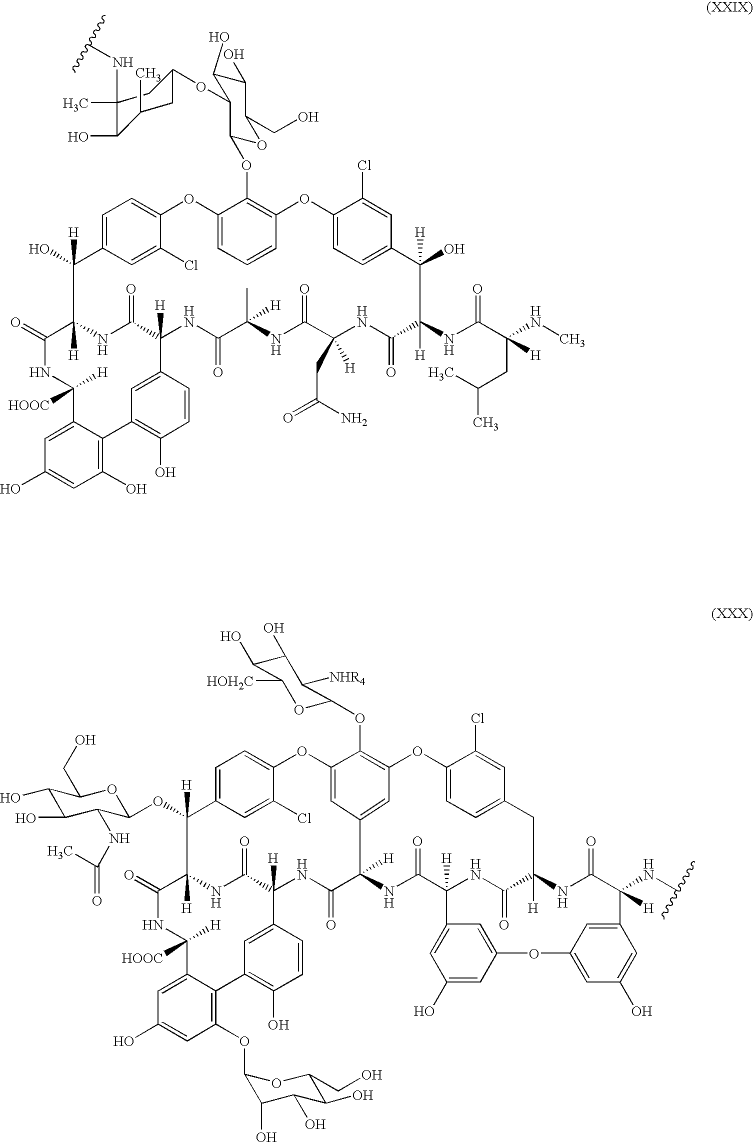
in which R₄ is as defined above.

The derivatives of vancomycin and of teicoplanin were for example fixed onto an aminoquinoline from one of their amino, carboxy, amide or hydroxyl type reactive functions.

In another family of compounds according to the invention, A represents a rifamycin residue such as those described by the formulae (XXXIa) and (XXXIb), as follows, 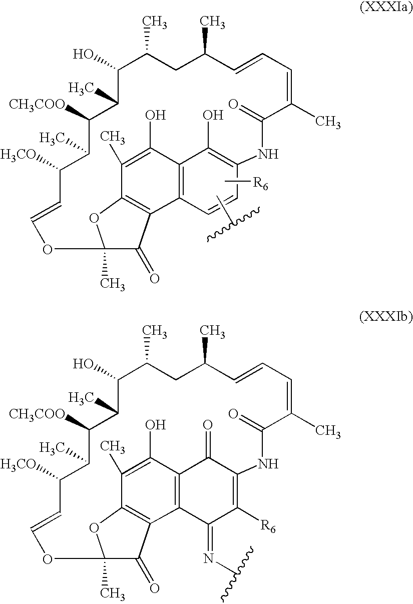
in which R₆ occupying any position and being able to form a cyclic structure with Y₁, Y₂ or U is as defined above.

The preparation of an aminoquinoline-rifamycin hybrid molecule was carried out for example from a reactive function of rifamycin of amino, halogen, hydroxy or aldehyde type.

In another embodiment of the compounds according to the invention, A represents a lipodepsipeptide residue such as the derivatives of daptomycin described by the following formula (XXXII),

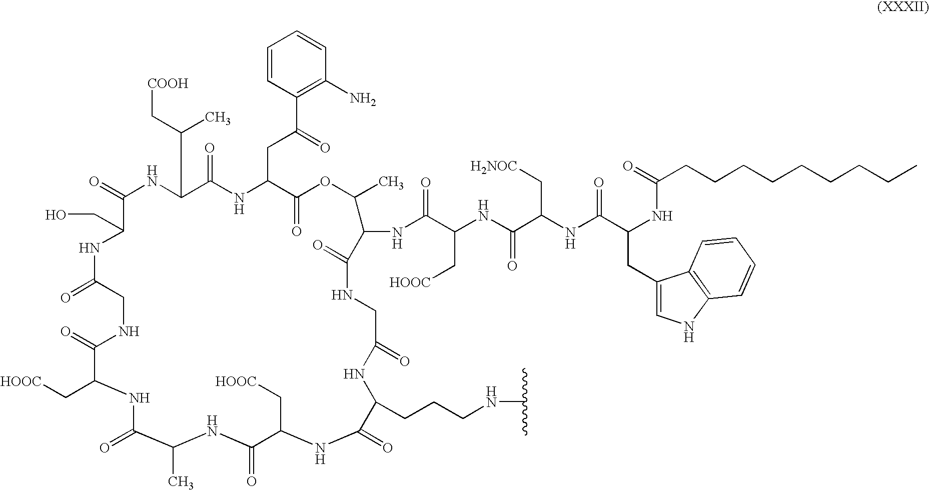

The lipodepsipeptides were grafted onto a quinoline for example from one of their amino, hydroxy or carboxy type reactive functions.

Furthermore, the aminoquinolines were covalently linked to an inhibitor of one of the biological systems developed by bacteria for inactivating the anti-microbial agents.

Inhibitors of resistance enzymes which are advantageously used within the context of the invention are inhibitors of β-lactamases, notably inhibitors of penicillinases, inhibitors of cephalosporinases, inhibitors of enzymes which inactivate aminosides, notably inhibitors of aminoside phosphotransferases (APH), aminoside adenylyltransferases (ANT), inhibitors of aminoside acetyltransferases (AAC), inhibitors of enzymes which inactivate M.L.S. (macrolide-lincosamide-streptogramins), or inhibitors of enzymes which inactivate phenicols.

Examples of these biological systems are advantageously p-lactamases, enzymes produced by certain bacteria which, by inactivating β-lactams, are for the most responsible for cases of resistance linked to these antibiotics. Advantageously, the inhibitors of p-lactamases are selected from classes A, B and C.

Advantageously, these inhibitors are clavulanic acid (3-(2-hydroxyethylidene)-7-oxo-4-oxa-1-azabicyclo[3.2.0]heptane-2-carboxylic acid), sulbactam sodium (3,3-dimethyl-7-oxo-4-thia-1-azabicyclo(3.2.0)-heptane-2-carboxylique acid 4,4 dioxide,[25-(2 alpha,5 alpha)]) and tazobactam sodium (2S-(2 alpha,3,beta,5 alpha)]-3-methyl-7-oxo-3-(1H-1,2,3-triazol-1-ylmethyl)-4-thia-1-azabicyclo[3.2.0]heptane-2-carboxylate 4,4-dioxide sodium)

The invention thus also covers the hybrid molecules of formula (I) for which A represents a residue which is an inhibitor of these biological systems, such as those described by the following formulae (XXXIII) to (XXXV), 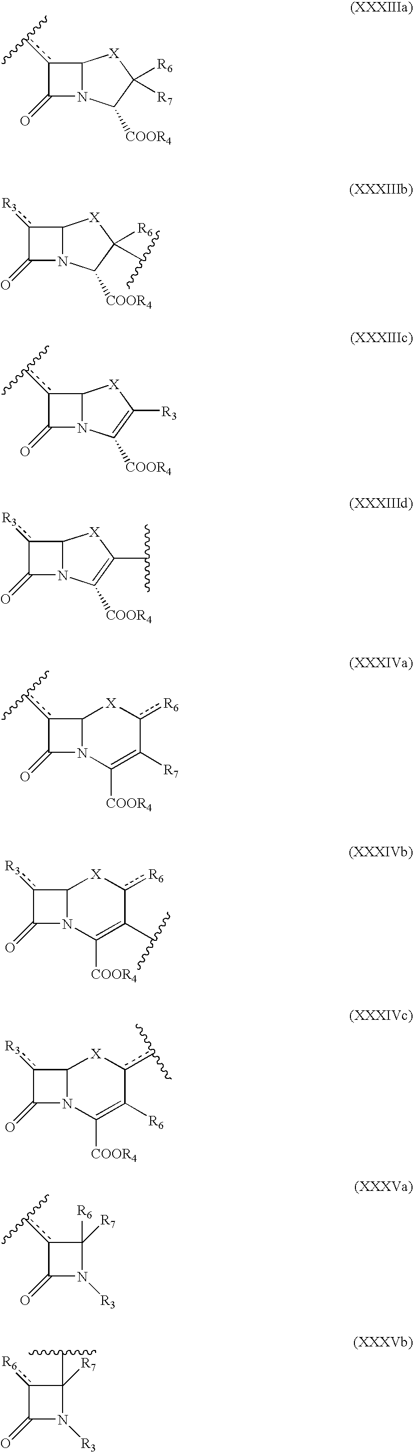 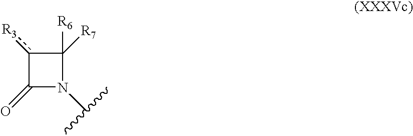
in which
R₃, R₄, R₆ and R₇ are as defined above,
X is an oxygen or sulfur atom or a group which is selected from S(O)ₘ, m being as defined above, or C(R₅ₐR_{5b}), R₅ₐ and R_{5b}, which are identical or different, being able if need be to form, together, a cyclic structure or a multiple bond are as defined above.

The formulae (IV) to (XXXV) give examples of anchoring sites of a quinoline onto a residue A, but other fixation sites were envisaged on the compounds A. It is understood that the invention covers the hybrid molecules aminoquinoline-A which are linked via any fixation site.

The invention also covers any hybrid molecule of formula (I) which covalently links an aminoquinoline to a residue A having antibiotic activity, one of its derivatives or precursors or a resistance enzyme inhibitor other than those described by the formulae (IV) to (XXXV).

Since the compounds of the invention have several asymmetric centres, they can exist in several stereochemical forms. The invention covers racemic mixtures, as well as optically pure isomers of these molecules of formula (I), and even mixtures in any proportion of said optically pure isomers. The invention also covers the achiral molecules.

The compounds of the invention can be in the form of acid addition salts, base addition salts or zwitterions, as well as prodrugs or prodrug salts. The invention also covers these various forms and their mixtures.

In the hybrid molecules of formula (I) in accordance with the invention, the aminoquinolines of formula (IIa) are more specially preferred in which the amino group is in position 4 with respect to the endocyclic nitrogen atom (these are thus 4-aminoquinolines) or the aminoquinolines of formula (IIb) in which the amino group is in position 8 (8-aminoquinolines). These 4-aminoquinolines and 8-aminoquinolines are of the following formulae (XXXVIa) and (XXXVIb), 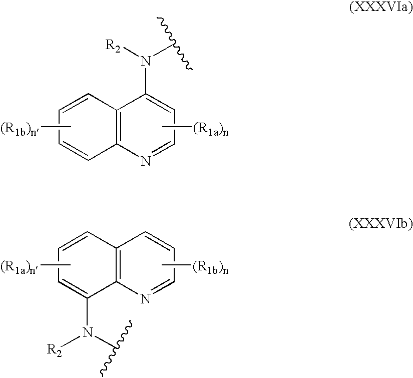
in which R₁ₐ, R_{1b}, R₂, n and n′ are as defined above.

Notably, an aim of the invention is hybrid molecules which correspond to the product of coupling comprising a 4-aminoquinoline of formula (XXXVIa) or an 8-aminoquinoline of formula (XXXVIb) and a residue A of the family of penicillins of formula (IV). Such molecules are of the structure (XXXVIIa) or (XXXVIIb) in which R₁ₐ, R_{1b}, R₂, R₃ₐ, R_{3b}, R₄, Y₁, Y₂, U, p, p′, p″, m, n and n′ are as defined above.

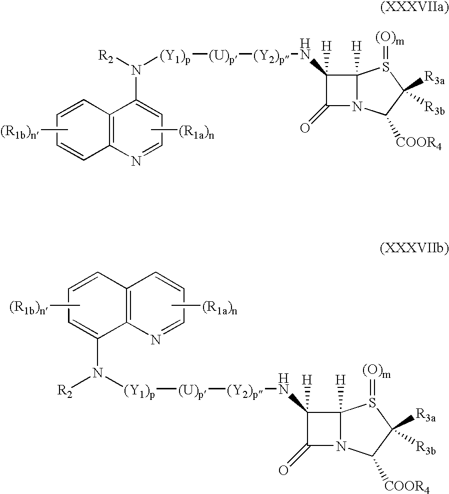

Other preferred hybrid molecules correspond to the coupling product comprising a 4-aminoquinoline of formula (XXXVIa) or an 8-aminoquinoline of formula (XXXVIb) and a residue A of the family of cephalosporins of formula (VIIIa). These important hybrid molecules are of the structure (XXXVIIIa) or (XXXVIIIb) in which R₁, R₂, R₃, R₄, Y₁, Y₂, U, p, p′, p″, m, n and n′ are as defined above.

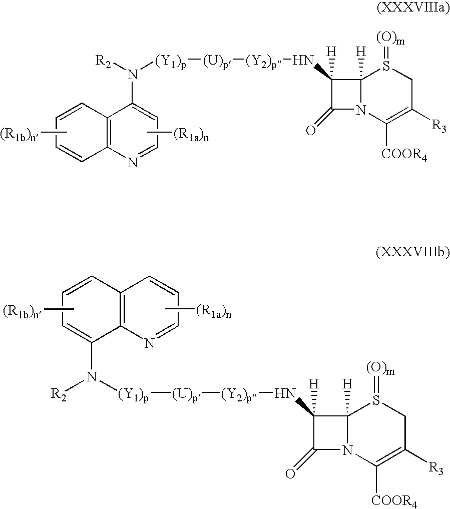

Another type of preferred compounds is characterized in that it relates to the aminoquinoline-quinolone molecules of formula (XXXIXa) or (XXXIXb) in which R₁, R₂, R₄, R₆, R₇, Y₁, Y₂, U, Z, p, p′, p″, n and n′ are as defined above.

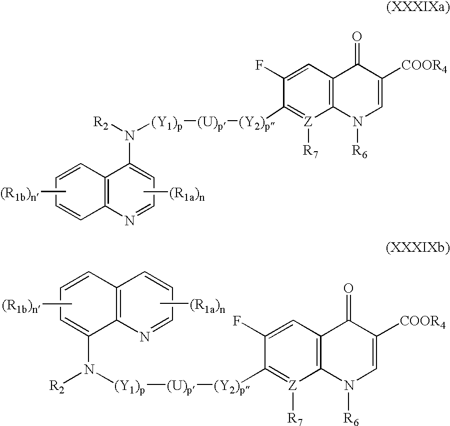

According to a preferred embodiment of the invention, R₄, in the various definitions of molecules given above, is a hydrogen atom, a protecting group (such as diphenylmethyl or allyl) or a group which is easily hydrolysable in vivo within the context of prodrug molecules (such as 2,2-dimethyl-propionyloxymethyl).

In another embodiment, R₁ advantageously represents a sole substituent, this substituent being a halogen atom or a methyl or trifluoromethyl group occupying any position.

In the hybrid molecules of aminoquinoline-penicillin or aminoquinoline-cephalosporin type of formulae (XXXVIIa), (XXXVIIb), (XXXVIIIa) or (XXXVIIIb),
according to a preferred embodiment, R₂ is a hydrogen atom or forms a cyclic structure with Y₁ including N of the aminoquinoline (such as a piperidine),
as (Y₁)ₚ—(U)_{p′}—(Y₂)_{p″} group, a group in which p=p′=1 and p″=0, Y₁ being as defined above, is preferred, and advantageously representing a methyl, ethyl, propyl or piperidine group (in including R₂ and N of the aminoquinoline) and U being as defined above and advantageously representing a carbonyl group. Compounds of formula (XXXVIIa), (XXXVIIb), (XXXVIIIa) and (XXXVIIIb) according to the invention which comprise a C1 to C6 alkylcarbonyl group, preferably acetyl, propionyl, butyryl, or piperidinecarbonyl (in the case in which R₂ and N are included) as (Y₁)ₚ—(U)_{p′}—(Y₂)_{p″} group are particularly preferred.

In the hybrid molecules of aminoquinoline-quinolone type of formulae (XXXIXa) and (XXXIXb),
according to a preferred embodiment, Z is a carbon atom and R₇ a hydrogen atom, R₆ is a C1 to C6 alkyl chain which is linear, branched or cyclic (preferably a cyclopropyl) and R₂ forms a cyclic structure with Y₂ including N of the aminoquinoline;
as (Y₁)ₚ—(U)_{p′}—(Y₂)_{p″} group, a group is preferred in which p=p′=0 and p″=1, Y₂ being as defined above and advantageously representing a C1 to C6 alkyl chain containing an amine moiety and forming a cyclic structure with R₂ including N of the aminoquinoline. Compounds of formula (XXXIXa) and (XXXIXb) according to the invention are particularly preferred, notably those which comprise a piperazine group as (Y₁)ₚ—(U)_{p′}—(Y₂)_{p″} group including N of the aminoquinoline and R₂

Advantageously, the compounds of formula (I) are those having the group Q representing a group of formula (IIa) or (IIb) defined above.

Advantageously, the compounds of formula (I) are those having the group A representing a group of formula (IV) defined above.

Advantageously, the compounds of formula (I) are those having the group A representing a group of formula (VIIIa) or (VIIIb) defined above.

Advantageously, the compounds of formula (I) are those having the group A representing a group of formula (XIII) defined above.

Advantageously, the compounds of formula (I) are those having the groups —(Y₁)ₚ—(U)_{p′}—(Y₂)_{p″}— representing a group in which p=p′=1 and p″=0, Y₁ being as defined above and advantageously representing a methyl, ethyl, propyl or piperidine group (in including R₂ and N of the aminoquinoline) and U being as defined above and advantageously representing a carbonyl group.

Advantageously, the compounds of formula (I) are those having the groups —(Y₁)ₚ—(U)_{p′}—(Y₂)_{p″}— representing a group in which p=p′=0 and p″=1, Y₂ being as defined above and advantageously representing a C1 to C6 alkyl chain containing an amine moiety and advantageously forming a cyclic structure with R₂ including N of the aminoquinoline.

Advantageously, according to the invention, A represents a cephalosporin, a penicillin or a quinolone.

The invention also covers methods of synthesis of the molecules of formula (I) defined above.

These methods comprise the reaction of reactive derivatives of aminoquinolines Q and of precursors of derivatives having antibiotic activity A, so as to form, between these derivatives, a coupling arm —(Y₁)ₚ—(U)_{p′}—(Y₂)_{p″}— as defined with respect to formula (I).

Various synthetic routes will be easily accessible to the person skilled in the art in proceeding according to classical techniques.

Advantageously, the method of preparing a compound Q-(Y₁)ₚ—(U)_{p′}—(Y₂)_{p″}-A, as defined above comprises:
a) either fixing the (Y₁)ₚ—(U)_{p′}—(Y₂)_{p″} group onto an aminoquinoline Q, and then reacting this intermediate compound with A, notably an antibiotic;
b) or fixing the (Y₁)ₚ—(U)_{p′}—(Y₂)_{p″} group with A, notably an antibiotic, and then fixing this intermediate onto an aminoquinoline Q;
c) or fixing an amino —(Y₁)ₚ—(U)_{p′}—(Y₂)_{p″} group onto a corresponding quinoline enabling an intermediate compound Q-(Y₁)ₚ—(U)_{p′}—(Y₂)_{p″} to be obtained, and then fixing this intermediate compound onto A, notably onto an antibiotic A.

Thus, as examples, in order to prepare hybrid molecules having a 4-aminoquinoline of formula (XXXVIa) as derivative Q and a penicillin of formula (IV) as residue A:
a-1) a compound of formula (XL): 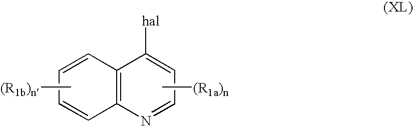
in which R₁ₐ and R_{1b} are as defined above and <<hal>> represents a halogen atom, is allowed to react with a derivative of formula (XLI):
R₂NH—(Y₁)ₚ—(U)_{p′} (XLI)
wherein R₂, Y₁, p and p′ are as defined above and U represents a carboxy or carboxyalkyl group (preferably U=COOH), affording a 4-aminoquinoline of formula (XLII): 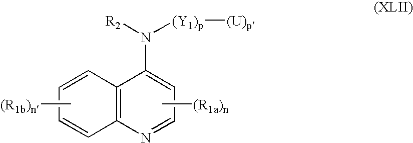
in which R₁ₐ, R_{1b}, R₂, Y₁, p and p′ are as defined above,
b-1) the coupling of the 4-aminoquinoline of formula (XLII) is then carried out in the presence of an activating agent of the U function, with a precursor of the antibiotic residue A of formula (XLIII), if need be as an addition salt with an acid (such as p-toluenesulfonic acid) in which R₃ₐ, R_{3b}, R₄, and m are as defined above, 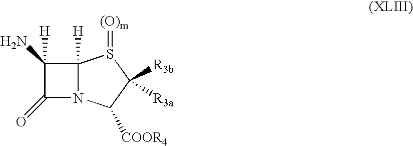
which leads to the hybrid molecules of formula (XXXVIIa) in which p″=0.

Step a-1) is advantageously carried out in molten phenol, at a temperature of 120° C. to 150° C. under stirring for 24 hours. After cooling, the product is obtained after various washings and/or extractions and, if need be, recrystallization by dissolution in aqueous carbonate and then precipitation by adding hydrochloric acid.

Step b-1) is advantageously carried out in a solvent such as an amide (preferably dimethylformamide) in the presence of an activating agent of the U function (PyBOP® or the dicyclohexylcarbodiimide/hydroxybenzotriazole system, for example) and under stirring for 24 hours at room temperature.

For the preparation of the hybrid molecules of formula (XXXVIIb) in which p″=0 and having an 8-aminoquinoline of formula (XXXVIb) as derivative Q:
b-2) the coupling reaction is carried out between a reactive derivative of 8-aminoquinoline of formula (XLIV) wherein R₁ₐ, R_{1b}, R₂, Y₁, p and p′ are as defined above and U represents a group carboxy or carboxyalkyl (preferably U=COOH): 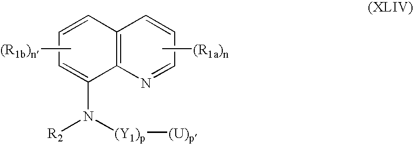
and a precursor of antibiotic residue A of formula (XLIII). This coupling reaction leads to the hybrid molecules of formula (XXXVIIb) in which p″=0.

Step b-2) is advantageously carried out according to the conditions described for step b-1) in the presence of an activating agent of the U function (PyBOP® or the dicyclohexylcarbodiimide/hydroxybenzotriazole system, for example).

In another method, in order to prepare hybrid molecules having a cephalosporin of formula (VIIIa) as residue A and an aminoquinoline of formula (XXXVIa) as derivative Q:
b-3) the coupling of the reactive derivative of aminoquinoline of formula (XLII) is carried out, in the presence of an activating agent of the U function, with a cephalosporin of formula (XLV), if need be as an addition salt with an acid (such as p-toluenesulfonic acid) in which R₃ and R₄ are as defined above and m=0. 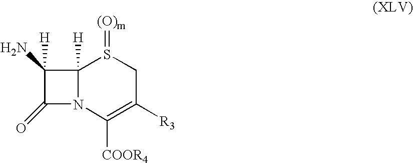
which leads to a mixture of isomers of Δ² and Δ³ cephems of formula (XLVI): 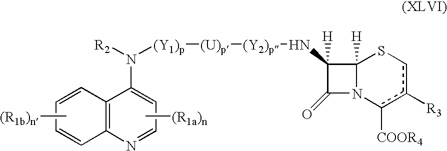
wherein R₁ₐ, R_{1b}, R₂, R₃, R₄, Y₁, U, p and p′ are as defined above,
c) an oxidation is then carried out of the mixture of isomers Δ²/Δ³ of formula (XLVI), which leads to the oxidised cephalosporins of A³ configuration alone, of formula (XLVII): 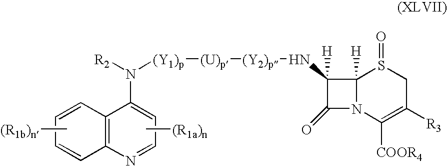
in which R₁ₐ, R_{1b}, R₂, R₃, R₄, Y₁, U, p and p′ are as defined above. This oxidation is followed if need be by an acid hydrolysis of the ester function COOR₄ for the synthesis of the hybrid molecules of formula (XXXVIIIa) in which R₄═H and m=1. These latter molecules can then be obtained as a salt by reaction with a pharmacologically acceptable acid,
d) the compounds of formula (XLVII) are reduced in order to obtain the hybrid molecules of formula (XXXVIIIa) in which R₁ₐ, R_{1b}, R₂, R₃, R₄, Y₁, U, p and p′ are as defined above and m=0. In the case in which R₄ is a protecting group, the deprotection can be carried out by acid hydrolysis. This step is followed if need be by a protonation with a pharmacologically acceptable acid, in order to obtain the product as a salt.

Step b-3) is advantageously carried out according to the conditions described for step b-1) in the presence of an activating agent of the U function (PyBOP® or the dicyclohexylcarbodiimide/hydroxybenzotriazole system for example).

Step c) is advantageously carried out in a halogenated solvent (dichloromethane for example) at 0° C. by slowly adding, over 3 hours, a solution of the oxidizing agent (for example 3-chloroperoxybenzoic acid).

Step d) is advantageously carried out at low temperature (−20° C.) over 1 hour in an amide solvent (dimethylformamide for example) under an inert atmosphere and in the presence of a reducing agent such as trichlorophosphine.

When a deprotection step is necessary, it is advantageously carried out in a halogenated solvent, under an inert atmosphere, in the presence of a compound used for trapping the carbocation released (anisole for example). The hydrolysis can be carried out by adding an acid (such as trifluoroacetic acid) at 0° C. extended with stirring for 1 hour and 30 minutes at room temperature.

In another method, in order to prepare aminoquinoline-quinolone hybrid molecules of formula (XXXIXa) in which p=p′=0, the coupling of a halogenated quinoline of formula (XL) is carried out with a quinolone derivative of formula (XLVIII): 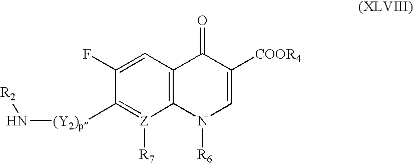
in which R₂, R₄, R₆, R₇, Y₂, Z and p″ are as defined above.

The coupling reaction is advantageously carried out in molten phenol at a temperature of 140° C. under stirring for 24 hours.

In order to obtain the hybrid molecules as an acid addition salt, the basic nitrogens are protonated by adding a pharmacologically acceptable acid. Salts formed with mineral acids (hydrochlorides, hydrobromides, sulfates, nitrates, phosphates) or with organic acids (citrates, tartrates, fumarates, lactates) can be cited as examples of addition salts with pharmacologically acceptable acids. The reaction can be carried out with 2 equivalents of acid added at 0° C.

The compounds of formula (I) can also be converted into metal salts or addition salts with nitrogen-containing bases according to methods known perse. Salts with alkali metals (sodium, potassium, lithium), or with alkaline-earth metals (magnesium, calcium), the ammonium salt or salts of nitrogen-containing bases (triethylamine, diisopropylamine, ethanolamine, procaine, N-benzyl-2-phenylethylamine, tris(hydroxymethyl)amino-methane, N,N′-dibenzylethylne-diamine), can be cited as examples of pharmacologically acceptable salts.

The invention also covers the prodrugs of the hybrid molecules of formula (I) which are hydrolysed in vivo releasing the active molecule. These prodrugs were prepared by the conventional techniques known to the person skilled in the art.

Advantageously, the invention covers the use of a compound Q as defined above for covalently fixing, for example via a —(Y₁)ₚ—(U)_{p′}—(Y₂)_{p″}— bond as defined above, an antibiotic residue A, one of its derivatives or precursors, or a resistance enzyme inhibitor, defined above.

The study of the pharmacological properties of the hybrid molecules of formula (XXXVIIa), (XXXVIIb) and (XXXVIIIa) given in the Examples has shown that these hybrid molecules are particularly interesting anti-microbial agents.

β-lactams are known for their activity on Gram+ germs. The hybrid molecules <<QA>> of formula (XXXVIIa), (XXXVIIb) and (XXXVIIIa) are very active in vitro on Staphylococcus aureus CIP 4.83 at minimum inhibitory concentrations (MIC) of between 0.01 and 0.78 μg/mL. Even more interesting is the activity of some of them on strains of Streptococcus pneumoniae DSP having decreased sensitivity to penicillin (CIP 104471 and a clinical isolate) at concentrations of between 0.006 and 6.25 μg/mL for the MIC and between 0.025 and 12.5 μg/mL for the MBC (minimum bactericidal concentration). The most active molecule (MIC: 0.006 μg/mL) proved to be 10 times more effective than ceftriaxone (MIC: 0.05 μg/mL), tested on the same stains. Ceftriaxone is one of the antibiotics which is currently used for treating cases of pneumonia which are due to S. pneumoniae germs which are resistant to penicillin. The hybrid molecules having an interesting activity on S. pneumoniae DSP (MIC of 0.006 to 0.39 μg/mL) have also proved to be active on Haemophilus influenzae, another germ which is responsible for pneumonia, with MIC values of 0.78 to 3.12 μg/mL.

Further, a study of stability in solution of these hybrid molecules has shown that they were stable not only at physiological pH, pH 7, in solution at 37° C., but also in acid medium at pH 1 (equivalent to the pH of the stomach). As an example, the half life of the molecule which is the most active on penicillin-resistant S. pneumoniae is 15 hours at pH 1 in solution at 37° C., while ceftriaxone is practically totally degraded under the same conditions in less than 6 hours, with a half life of less than 2 hours.

These properties render said products of the invention, as well as their salts, hydrates, prodrugs and prodrug salts, able to be used as medicaments.

The invention covers compositions, notably by taking advantage of the properties of these hybrid molecules, for the preparation of pharmaceutical compositions.

Notably, the pharmaceutical composition comprises, notably as active principle, at least one compound QA defined above, in a pharmaceutically acceptable excipient.

The pharmaceutical compositions of the invention contain an effective amount of at least one hybrid molecule of formula (I) as defined above, as well as a pharmaceutically acceptable vehicle. As is known to the person skilled in the art, various forms of excipients can be used adapted to the mode of administration and some of them can promote the effectiveness of the active molecule, e.g. by promoting a release profile rendering this active molecule overall more effective for the treatment desired.

The pharmaceutical compositions of the invention are thus able to be administered in various forms, more specially for example in an injectable, pulverisable or ingestible form, for example via the intramuscular, intravenous, subcutaneous, intradermal, oral, topical, rectal, vaginal, ophthalmic, nasal, transdermal or parenteral route. The present invention notably covers the use of a compound according to the present invention for the manufacture of a composition, particularly a pharmaceutical composition.

The compositions of the invention are particularly appropriate for treating a bacterial infection in man or in an animal or for disinfecting materials, notably medical materials.

The invention also covers the use of a compound according to the present invention for the manufacture of a composition, notably a pharmaceutical composition, which is intended notably for treating medical material which is contaminated by bacteria, or for treating a bacterial infection of an animal, particularly a human being, due to Gram+ and/or Gram− bacteria, preferably due to Staphylococcus aureus, Enterococcus faecalis, for example vancomycin-resistant Enterococcus faecalis, Streptococcus pneumoniae, for example Streptococcus pneumoniae having decreased sensitivity to penicillins or Haemophilus influenzae.

Thus, the hybrid molecules of the invention can very advantageously be used for treating infections due to strains of Streptococcus pneumoniae which are sensitive or resistant to penicillin, such as pneumonia, meningitis, otitis, or acute sinusitis. By virtue of their activity on Staphylococcus aureus, it is possible for the hybrid molecules to be used for treating infections due to strains of this bacterium, such as infections of the skin and of the skin structure, nosocomial infections, or osteomyelitis. The invention thus also covers the application of these very important hybrid molecules defined above for preparing medicaments which are intended for the food industry, and in human and veterinary medicine for treating a bacterial infection or as a bactericide for industrial applications.

Notably, it is advantageous to deliver an effective amount of the compound of the present invention for the treatments which are cited above and below.

The invention even covers a method of therapeutic treatment of an animal or of a human being in need thereof, characterized in that it comprises administering to it a therapeutically effective amount of a hybrid compound according to the invention of formula (I) mentioned above. Particular embodiments of this treatment result clearly for the person skilled in the art from the activity of the antibiotics concerned and from the description of the invention taken in its entirety and including the Examples which make up an integral part of it.

The invention is now illustrated by Examples which represent currently preferred embodiments which make up a part of the invention but which in no way are to be used to limit the scope of it, the invention being pioneer within the context of the creation of a novel family of active compounds covalently combining at least one antibiotic and at least one aminoquinoline.

In the Examples, all the percentages are given by weight (unless indicated otherwise), the temperature is in degrees Celsius, the pressure is atmospheric pressure, unless indicated otherwise.

### EXAMPLES

Examples 1 to 4 below exemplify preparations of hybrid molecules of the family of aminoquinolines-penicillins (peniciquines).

### Example 1

### Preparation of an Aminoquinoline-Penicillin Hybrid, ref PA 1007

### (2S,5R,6R)-6-{[1-(7-Chloro-quinolin-4-yl)-piperidine-4-carbonyl]-amino}-3,3-dimethyl-7-oxo-4-thia-1-aza-bicyclo[3.2.0]heptane-2-carboxylic acid 2,2-dimethyl-propionyloxymethyl ester

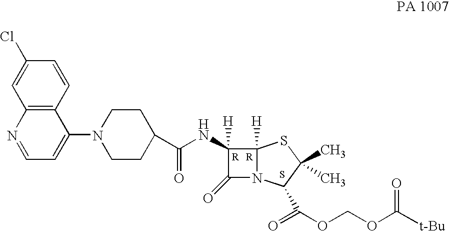

### 1.1. 1-(7-Chloro-quinolin-4-yl)-piperidine-4-carboxylic acid

A mixture of 4,7-dichloroquinoline (17.4 g, 0.09 mol), of isonipecotic acid (23.8 g, 0.18 mol) and phenol (46.3 g, 0.49 mol) is heated at 120° C. under magnetic stirring for 24 hours. After cooling, the reaction mixture is diluted with 400 mL of ethyl acetate, filtered and the precipitate is washed with water. This precipitate is then recrystallized by dissolution in 300 mL of hot (100C) 10% (w/v) aqueous Na₂CO₃ and precipitation at 0° C. by addition of 2M HCl until pH 5. The precipitate formed is filtered off and then washed successively with water, acetone and diethyl ether before being dried under vacuum. The product is obtained as a white powder (18.4 g, 72%).

¹H NMR (300 MHz, CD₃COOD) δ ppm: 2.11 (2H, dd, J=10.6 Hz, J=13.9 Hz), 2.27 (2H, d, J=13.9 Hz), 2.92 (1H, m), 3.60 (2H, dd, J=10.6 Hz, =13.4 Hz), 4.20 (2H, d, J=13.4 Hz), 7.19 (1H, d, J=7.0 Hz), 7.65 (1H, dd, J=2.0 Hz, 1=9.2 Hz), 8.10 (1H, d, J=9.2 Hz), 8.18 (1H, d, J=2.0 Hz), 8.72 (1H, d, J=7.0 Hz). MS (IS>0) m/z: 291.0 (M+H⁺).

### 1.2. (2S,5R,6R)-6-{[1-(7-Chloro-quinolin-4-yl)-piperidine-4-carbonyl]-amino}-3,3-dimethyl-7-oxo-4-thia-1-aza-bicyclo[3.2.0]heptane-2-carboxylic acid 2,2-dimethyl-propionyl-oxymethyl ester

3.6 mL of N-methylmorpholine (32.7 mmol) are added to a mixture of 1-(7-chloro-quinolin-4-yl)-piperidine-4-carboxylic acid (Example 1.1) (3.0 g, 10.3 mmol) and 6-aminopenicillanic acid pivaloyloxymethyl ester tosylate salt POM-APA-Ts (prepared according to the method described by R.-de-Vains et al., Tetrahedron Lett. 2001, 42, 7033-7036) (5.2 g, 10.3 mmol) in 75 mL of DMF. The suspension is left under magnetic stirring 15 minutes before addition of the activating agent PyBOP® (5.4 g, 10.3 mmol). The magnetic stirring is continued for 24 hours at room temperature. The reaction mixture is then diluted with 100 mL of dichloromethane and then washed successively with 100 mL of 10% (w/v) aqueous Na₂CO₃, twice 100 mL of water and 100 mL of water saturated with NaCl. The organic phase is dried over magnesium sulfate, filtered and then evaporated. The oil obtained is purified by liquid chromatography on silica gel (SiO₂ 60A C.C 70-200 μm, eluent: ethyl acetate). The cleanest fractions on TLC revealed under UV are evaporated. PA 1007 is obtained after recrystallization from chloroform/n-hexane as a white powder (1.4 g, 23%).

IR (KBr) cm⁻¹: (C═O) 1786, 1757, 1681, ¹H NMR (300 MHz, CDCl₃) δ ppm: 1.22 (9H, s), 1.53 (3H, s), 1.65 (3H, s), 2.13 (4H, m), 2.43 (1H, m), 2.84 (2H, dd, J=11.4 Hz, J=12.3 Hz), 3.60 (2H, d, J=12.3 Hz), 4.44 (1H, s), 5.58 (1H, d, J=4.0 Hz), 5.75 (1H, dd, J=4.0 Hz, J=8.7 Hz), 5.77 (1H, d, J=5.7 Hz), 5.88 (1H, d, J=5.7 Hz), 6.57 (1H, d, J=8.7 Hz), 6.80 (1H, d, J=5.1 Hz), 7.41 (1H, dd, J=2.0 Hz, J=9.0 Hz), 7.89 (1H, d, J=9.0 Hz), 8.02 (1H, d, J=2.0 Hz), 8.69 (1H, d, J=5.1 Hz). MS (IS>0) m/z: 603.2 (M+H⁺). Anal. calcd. for C₂₉H₃₅ClN₄O₆S.0.5H₂O: C, 56.90; N, 9.15; found: C, 56.80; N, 8.83.

### Example 2

### Preparation of an Aminoquinoline-Penicillin Hybrid, ref PA 1008

### (2S,5R,6R)-3,3-Dimethyl-7-oxo-6-[3-(quinolin-8-ylamino)-propionyl-amino]-4-thia-1-aza-bicyclo[3.2.0]heptane-2-carboxylic acid 2,2-dimethyl-propionyloxymethyl ester

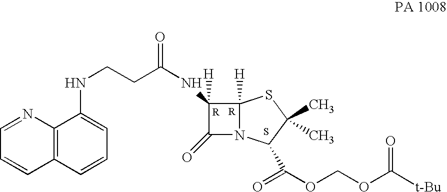

PA 1008 is prepared according to the procedure described in Example 1.2 from 4.3 g of 3-(quinolin-8-ylamino)-propionic acid (19.9 mmol) (prepared according to the method described by Z. J. Beresnevicius et al., Chem. Heterocycl. Comp. 2000, 36, 432-438), 10.0 g of POM-APA-Ts (19.9 mmol), 6.5 mL of N-methylmorpholine (59.1 mmol) and 10.3 g of PyBOP® (19.9 mmol). PA 1008 is obtained after purification by liquid chromatography on silica gel (SiO₂ 60A C.C 6-35 μm, eluent: n-hexane/ethyl acetate 55/45 v/v) and recrystallization from diethyl ether/n-hexane as a yellow powder (2.3 g, 22%).

IR (KBr) cm⁻¹: (C═O) 1784, 1755, 1667. ¹H NMR (300 MHz, 298K, CDCl₃) 6, ppm: 1.16 (9H, s), 1.37 (6H, s), 2.64 (2H, t, J=6.6 Hz), 3.61 (2H, m), 4.34 (1H, s), 5.45 (1H, d, J=4.2 Hz), 5.67 (1H, dd, J=4.2 Hz, J=8.7 Hz), 5.70 (1H, d, J=5.4 Hz), 5.80 (1H, d, J=5.4 Hz), 6.34 (1H, broad s), 6.67 (1H, d, J=7.5 Hz), 7.03 (1H, d, J=8.4 Hz), 7.09 (1H, d, J=8.7 Hz), 7.30 (1H, dd, J=4.2 Hz, 1=8.1 Hz), 7.32 (1H, dd, J=7.5 Hz, J=8.4 Hz), 7.99 (1H, dd, J=1.5 Hz, J=8.1 Hz) 8.66 (1H, dd, J=1.5 Hz, J=4.2 Hz). MS (IS>0) m/z: 529.2 (M+H⁺). Anal. calcd. for C₂₆H₃₂N₄O₆S: C, 59.07; N, 10.60; found: C, 59.19; N, 10.50.

### Example 3

### Preparation of an Aminoquinoline-Penicillin Hybrid, ref PA 1012

### (2S,5R,6R)-6-[2-(7-Chloro-quinolin-4-ylamino)-acetylamino]-3,3-dimethyl-7-oxo-4-thia-1-aza-bicyclo[3.2.0]heptane-2-carboxylic acid 2,2-dimethylpropionyloxymethyl ester

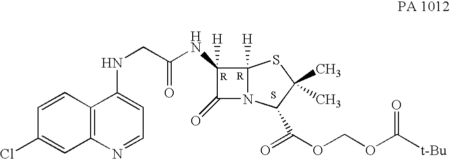

### 3.1. (7-Chloro-quinolin-4-ylamino)-acetic acid

This compound is prepared by modification of the method described by E. O. Titus et al. (J. Org. Chem, 1948. 13, 61). A mixture of 4,7-dichloroquinoline (30.0 g, 0.15 mol), glycine (25.0 g, 0.33 mol) and phenol (80.0 g, 0.85 mol) is heated at 120° C. under magnetic stirring for 24 hours. After cooling, the reaction mixture is diluted with 1 L of diethyl ether and extracted with 1 L of 10% (w/v) aqueous Na₂CO₃. The aqueous phase is warmed with decolorizing carbon (Norit A) at 100° C., filtered and neutralized to pH 5 at 0° C. with 2 M aqueous HCl. The precipitate formed is filtered off and washed successively with water, acetone and diethyl ether before being dried under vacuum. The product is obtained as a white powder (27.0 g, 75%).

¹H NMR (300 MHz, CF₃COOD) δ ppm: 4.51 (2H, s), 6.72 (1H, d, 7=6.9 Hz), 7.68 (1H, d, J=9.0 Hz), 7.87 (1H, s), 8.10 (1H, d, J=9.0 Hz), 8.30 (1H, d, J=6.9 Hz).

### 3.2. (2S,5R,6R)-6-[2-(7-Chloro-quinolin-4-ylamino)-acetylamino]-3,3-dimethyl-7-oxo-4-thia-1-aza-bicyclo[3.2.0]heptane-2-carboxylic acid 2,2-dimethyl-propionyloxymethyl ester

PA 1012 is prepared according to the procedure described in Example 1.2 from 1.3 g of (7-chloro-quinolin-4-ylamino)-acetic acid (Example 3.1) (5.6 mmol), 2.8 g of POM-APA-Ts (5.6 mmol), 1.8 mL of N-methylmorpholine (16.4 mmol) and 2.9 g of PyBOP® (5.6 mmol). PA 1012 is obtained after purification by liquid chromatography on silica gel (SiO₂ 60A C.C 70-200 μm, eluent: ethyl acetate/chloroform 8/2 v/v) and recrystallization from chloroform/n-hexane as a white powder (0.3 g, 11%).

IR (KBr) cm⁻¹: (C═O) 1784, 1759, 1669. ¹H NMR (300 MHz, CDCl₃) δ ppm: 1.20 (9H, s), 1.39 (3H, s), 1.44 (3H, s), 4.04 (2H, broad s), 4.39 (1H, s), 5.57 (1H, d, J=4.2 Hz), 5.74 (1H, dd, J=4.2 Hz, J=9.0 Hz), 5.75 (1H, d, J=5.4 Hz), 5.85 (1H, d, J=5.4 Hz), 6.21 (1H, broad s), 6.29 (1H, d, J=6.0 Hz), 7.36 (1H, dd, J=1.8 Hz, J=9.0 Hz), 7.53 (1H, d, J=9.0 Hz), 7.77 (1H, d, J=9.0 Hz), 7.95 (1H, d, J=1.8 Hz), 8.51 (1H, d, J=6.0 Hz). MS (IS>0) m/z: 549.2 (M+H⁺). Anal. calcd. for C₂₅H₂₉ClN₄O₆S.1.5H₂O: C, 52.12; N, 9.72; found: C, 52.41; N, 9.39.

### Example 4

### Preparation of an Aminoquinoline-Penicillin Hybrid, ref PA 1013

### (2S,5R,6R)-6-[3-(7-Chloro-quinolin-4-ylamino)-propionylamino]-3,3-dimethyl-7-oxo-4-thia-1-aza-bicyclo[3.2.0]heptane-2-carboxylic acid 2,2-dimethyl-propionyloxymethyl ester

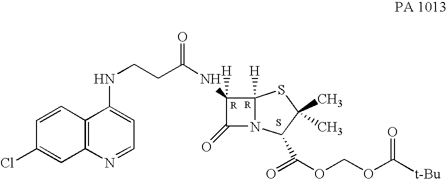

### 4.1. 3-(7-Chloro-quinolin-4-ylamino)-propionic acid

This compound is prepared by modification of the method described by W. J. Humphlett et al. (I. Am. Chem. Soc. 1951, 73, 61), according to the procedure described in Example 3.2 and from 25.1 g of 4,7-dichloroquinoline (0.13 mol), 23.6 g of β-alanine (0.26 mol) and 66.5 g of phenol (0.71 mol). The product is obtained as a white powder (19.5 g, 62%).

¹H NMR (300 MHz, CF₃COOD) δ ppm: 2.90 (2H, t, J=6.0 Hz), 3.86 (2H, t, J=6.0 Hz), 6.73 (1H, d, J=7.2 Hz), 7.53 (1H, dd, J=1.5 Hz, =9.0 Hz), 7.72 (1H, d, J=1.5 Hz), 7.96 (1H, d, J=9.0 Hz), 8.14 (1H, d, J=7.2 Hz).

### 4.2. (2S,5R,6R)-6-[3-(7-Chloro-quinolin-4-ylamino)-propionylamino]-3,3-dimethyl-7-oxo-4-thia-1-aza-bicyclo[3.2.0]heptane-2-carboxylic acid 2,2-dimethyl-propionyloxymethyl ester

PA 1013 is prepared according to the procedure described in Example 1.2 from 2.2 g of 3-(7-chloro-quinolin-4-ylamino)-propionic acid (Example 4.1) (8.0 mmol), 4.1 g of POM-APA-Ts (8.0 mmol), 2.6 mL of N-methylmorpholine (23.6 mmol) and 4.1 g of PyBOP® (8.0 mmol). PA 1013 is obtained after several recrystallizations from chloroform/n-hexane as a white powder (1.2 g, 27%).

IR (KBr) cm⁻¹: (C═O) 1787, 1760, 1662. ¹H NMR (300 MHz, CDCl₃) δ ppm: 1.23 (9H, s), 1.48 (3H, s), 1.53 (3H, s), 2.73 (2H, m), 3.69 (2H, m), 4.42 (1H, s), 5.55 (1H, d, J=4.2 Hz), 5.71 (1H, dd, J=4.2 Hz, J=8.7 Hz), 5.77 (1H, d, J=5.7 Hz), 5.87 (1H, d, J=5.7 Hz), 6.37 (1H, d, J=5.4 Hz), 6.75 (1H, broad s), 7.37 (1H, dd, J=1.8 Hz, J=9.0 Hz), 7.76 (1H, d, J=9.0 Hz), 7.93 (1H, d, J=1.8 Hz), 8.46 (1H, d, J=5.4 Hz). MS (IS>0) m/z: 563.3 (M+H⁺). Anal. calcd. for C₂₆H₃₁ClN₄O₆S.0.5H₂O: C, 54.58; N, 9.79; found C, 54.41; N, 9.84.

Examples 5 to 16 exemplify preparations of hybrid molecules of the family of aminoquinolines-cephalosporins (cephaloquines).

### Example 5

### Preparation of an Aminoquinoline-Cephalosporin Hybrid, ref PA 1046

### (6R,7R)-3-Acetoxymethyl-7-[2-(7-chloro-quinolin-4-ylamino)-acetylamino]-8-oxo-5-thia-1-aza-bicyclo[4.2.0]oct-2-ene-2-carboxylic acid

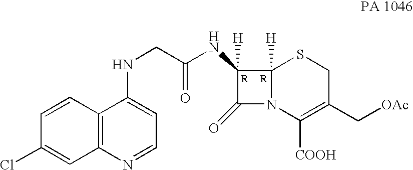

### 5.1. (6R,7R)-3-Acetoxymethyl-7-amino-8-oxo-5-thia-1-aza-bicyclo[4.2.0]oct-2-ene-2-carboxylic acid benzhydryl ester p-toluene sulfonic acid

This compound is prepared by modification of the method described by R. G. Micetich et al. (Synthesis 1985, 6-7, 693-695). A solution of diphenyldiazomethane (0.133 mol) (freshly prepared according to the method described by S. Kumar et al., 3. Am. Chem. Soc. 1984, 106, 1040-1045) in 300 mL of dichloromethane is added dropwise over 2 hours to a solution of 7-aminocephalosporanic acid (7-ACA) (22.6 g, 0.08 mol) in 80 mL of methanol. The mixture is stirred at room temperature for 72 hours and then filtered. The filtrate is washed successively with 500 mL of 5% (w/v) aqueous Na₂CO₃, twice 500 mL of water and 500 mL of water saturated with NaCl. The organic phase is dried over magnesium sulfate, filtered and then evaporated. The resulting oil is dissolved in a minimum of dichloromethane and diluted with 200 mL of ethyl acetate. A solution of p-toluenesulfonic acid (15.7 g, 0.08 mol) in 200 mL of ethyl acetate is added dropwise to this solution under magnetic stirring. The product precipitates slowly as a tosylate and after 1 hour of stirring, the precipitate is filtered off, washed with ethyl acetate and then dried under vacuum. The product is obtained as a beige powder (29.0 g, 58%).

¹H NMR (300 MHz, CDCl₃) 8 ppm: 1.97 (3H, s), 2.27 (3H, s), 3.09 (1H, d, J=16.5 Hz), 3.32 (1H, d, J=16.5 Hz), 4.78 (1H, d, J=14.7 Hz), 4.79 (1H, d, J=3.6 Hz), 4.95 (1H, d, J=3.6 Hz), 5.17 (1H, d, J=14.7 Hz), 6.91 (1H, s), 7.05 (2H, d, J=7.8 Hz), 7.15-7.32 (8H, m), 7.39 (2H, d, J=7.8 Hz), 7.78 (2H, d, J=6.9 Hz), 8.83 (2H, broad s).

### 5.2. Mixture of (6R,7R)-3-acetoxymethyl-7-[2-(7-chloro-quinolin-4-ylamino)-acetylamino]-8-oxo-5-thia-1-aza-bicyclo[4.2.0]oct-2-ene-2-carboxylic acid benzhydryl ester and (6R,7R)-3-acetoxymethyl-7-[2-(7-chloro-quinolin-4-ylamino)-acetylamino]-8-oxo-5-thia-1-aza-bicyclo-[4.2.0]oct-3-ene-2-carboxylic acid benzhydryl ester: Δ²/Δ³

1-hydroxybenzotriazole HOBT (1.4 g, 10.4 mmol) and N,N′-dicyclohexylcarbodiimide DCC (2.1 g, 10.4 mmol) are added successively to a suspension of (7-chloro-quinolin-4-ylamino)-acetic acid (Example 3.1) (2.9 g, 10.0 mmol) in 80 mL of DMF. The mixture is stirred for 30 minutes before the addition of (6R,7R)-3-acetoxymethyl-7-amino-8-oxo-5-thia-1-aza-bicyclo[4.2.0]oct-2-ene-2-carboxylic acid benzhydryl ester p-toluenesulfonic acid (Example 5.1) (6.1 g, 10.0 mmol) followed by triethylamine (2.7 mL, 20.0 mmol). The mixture is stirred for additional 24 hours at room temperature. The reaction mixture is then diluted with 400 mL of ethyl acetate and then filtered. The filtrate is washed successively with 400 mL of 10% (w/v) aqueous Na₂CO₃, twice 400 mL of water and 400 mL of water saturated with NaCl. The organic phase is dried over magnesium sulfate, filtered and then evaporated. The resulting oil is purified by liquid chromatography on silica gel (SiO₂ 60A C.C 6-35 μm, eluent: dichloromethane/ethanol 90/10 v/v). The cleanest fractions on TLC revealed under UV are evaporated. The coupling product is obtained as a orangey powder (3.2 g, 48%) as a Δ²/Δ³ 37/63 mixture, used as such in the following step.

### 5.3. (6R,7R)-3-Acetoxymethyl-7-[2-(7-chloro-quinolin-4-ylamino)-acetylamino]-5,8-dioxo-5×4-thia-1-aza-bicyclo[4.2.0]oct-2-ene-2-carboxylic acid benzhydryl ester

A solution of 3-chloroperoxybenzoic acid (2.6 g, 15.1 mmol) in 250 mL of dichloromethane is added dropwise, over a period of 3 hours, to a solution of the Δ²/Δ³ mixture of Example 5.2 (5.1 g, 7.8 mmol) in 200 mL of dichloromethane, at 0° C. The reaction mixture is then washed with a mixture of 400 mL of 5% (w/v) aqueous Na₂CO₃ and 250 mL of 6% (w/v) aqueous sodium sulfite. The organic phase is dried over magnesium sulfate, filtered and then evaporated. The resulting solid is washed with ethyl acetate under magnetic stirring for 30 minutes, filtered, washed with diethyl ether and dried under vacuum. The oxidation product is obtained as a yellow powder (4.0 g, 76%).

IR (KBr) cm⁻¹: (C═O) 1788, 1738, 1663. ¹H NMR (300 MHz, DMSO) δ ppm: 1.95 (3H, s), 3.60 (1H, d, J=18.9 Hz), 3.93 (1H, d, J=18.9 Hz), 4.11 (2H, m), 4.58 (1H, d, J=13.2 Hz), 4.95 (1H, d, J=4.5 Hz), 5.02 (1H, d, J=13.2 Hz), 6.03 (1H, dd, J=4.5 Hz, J=8.1 Hz), 6.39 (1H, d, J=5.4 Hz), 6.94 (1H, s), 7.28-7.52 (11H, m), 7.83 (2H, broad s), 8.23 (1H, d, J=9.0 Hz), 8.34 (1H, d, J=8.1 Hz), 8.44 (1H, d, J=5.4 Hz). MS (IS>0) m/z: 673.1 (M+H⁺).

### 5.4. (6R,7R)-3-Acetoxymethyl-7-[2-(7-chloro-quinolin-4-ylamino)-acetylamino]-8-oxo-5-thia-1-aza-bicyclo[4.2.0]oct-2-ene-2-carboxylic acid benzhydryl ester

1.1 mL of trichlorophosphine (12.6 mmol) is added dropwise to a solution, at −20° C. under argon, of (6R,7R)-3-acetoxymethyl-7-[2-(7-chloro-quinolin-4-ylamino)-acetylamino]-5,8-dioxo-5×4-thia-1-aza-bicyclo[4.2.0]-oct-2-ene-2-carboxylic acid benzhydryl ester (Example 5.3) (3.8 g, 5.6 mmol) in 40 mL of dry DMF. The reaction is left under magnetic stirring for 1 hour at −20° C. The mixture is then diluted with 150 mL of dichloromethane and then washed successively with twice 150 mL of water and 150 mL of water saturated with NaCl. The organic phase is dried over magnesium sulfate, filtered and then evaporated. The product is obtained after recrystallization from dichloromethane/diethyl ether as a beige powder (1.7 g, 46%).

IR (KBr) cm⁻¹: (C═O) 1785, 1735, 1689. ¹H NMR (300 MHz, DMSO) δ ppm: 1.96 (3H, s), 3.56 (1H, d, J=18.3 Hz), 3.69 (1H, d, J=18.3 Hz), 4.37 (2H, m), 4.64 (1H, d, J=13.2 Hz), 4.86 (1H, d, J=13.2 Hz), 5.16 (1H, d, J=5.1 Hz), 5.83 (1H, dd, J=5.1 Hz, J=8.4 Hz), 6.71 (1H, d, J=7.2 Hz), 6.93 (1H, s), 7.27-7.49 (10H, m), 7.82 (1H, dd, J=1.8 Hz, J=9.0 Hz), 8.08 (1H, d, J=1.8 Hz), 8.57 (1H, d, J=9.0 Hz), 8.61 (1H, d, J=7.2 Hz), 9.38 (1H, d, J=8.4 Hz), 9.74 (1H, broad s). MS (IS>0) m/z: 657.2 (M+H⁺).

### 5.5. (6R,7R)-3-Acetoxymethyl-7-[2-(7-chloro-quinolin-4-ylamino)-acetylamino]-8-oxo-5-thia-1-aza-bicyclo[4.2.0]oct-2-ene-2-carboxylic acid

0.8 mL of anisole (7.5 mmol), followed by 1.4 mL of trifluoroacetic acid injected dropwise (19.0 mmol), is added to a solution, at 0° C. under argon, of (6R,7R)-3-acetoxymethyl-7-[2-(7-chloro-quinolin-4-ylamino)-acetylamino]-8-oxo-5-thia-1-aza-bicyclo[4.2.0] oct-2-ene-2-carboxylic acid benzhydryl ester (1.3 g, 1.9 mmol) (Example 5.4) in 15 mL of dry dichloromethane. The reaction is stirred for 1 hour and 30 minutes at room temperature. The product, as a triflate salt, is precipitated by adding diethyl ether and filtered off. The resulting solid is washed with water, ethanol, diethyl ether before being dried under vacuum. PA 1046 is obtained as a clear beige powder (0.5 g, 54%).

IR (KBr) cm⁻¹: (C═O) 1760, 1664. ¹H NMR (400 MHz, DMSO) δ ppm: 2.01 (3H, s), 3.22 (1H, d, J=17.6 Hz), 3.47 (1H, d, J=17.6 Hz), 4.05 (2H, d, J=6.0 Hz), 4.76 (1H, d, J=12.0 Hz), 4.97 (1H, d, J=4.8 Hz), 4.99 (1H, d, J=12.0 Hz), 5.51 (1H, dd, J=4.8 Hz, J=8.4 Hz), 6.33 (1H, d, J=5.6 Hz), 7.49 (1H, dd, 1=2.4 Hz, J=9.2 Hz), 7.80 (1H, d, J=6.0 Hz), 7.82 (1H, d, J=2.4 Hz), 8.25 (1H, d, J=9.2 Hz), 8.40 (1H, d, J=5.6 Hz), 9.11 (1H, d, J=8.4 Hz). MS (IS>0) m/z: 491.2 (M+H⁺). Anal. calcd. for C₂₁H₁₉ClN₄O₆S.2H₂O: C, 47.86; N, 10.63; found: C, 47.96; N, 10.36.

### Example 6

### Preparation of an Aminoquinoline-Cephalosporin Hybrid, ref PA 1089

### (6R,7R)-3-Acetoxymethyl-7-[2-(7-chloro-quinolin-4-ylamino)-acetylamino]-8-oxo-5-thia-1-aza-bicyclo[4.2.0]oct-2-ene-2-carboxylic acid hydrochloride

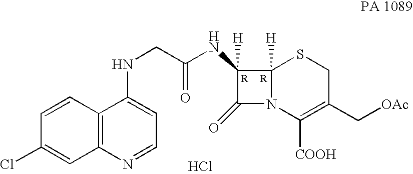

0.8 mL of 5 M HCl in 2-propanol (4.0 mmol) is added dropwise to a solution, at 0° C., of PA 1046 (Example 5.5) (0.5 g, 1.0 mmol) in 40 mL of a chloroform/ethanol mixture 1/1 v/v. After stirring for 30 minutes at 0° C., the product is precipitated by addition of diethyl ether. The precipitate is filtered off, washed with cold ethanol, then with diethyl ether and dried under vacuum. PA 1089 is obtained as a clear beige powder (0.4 g, 76%).

¹H NMR (300 MHz, DMSO) δ ppm: 2.03 (3H, s), 3.50 (1H, d, J=18.3 Hz), 3.65 (1H, d, J=18.3 Hz), 4.36 (2H, m), 4.70 (1H, d, J=12.9 Hz), 5.00 (1H, d, J=12.9 Hz), 5.12 (1H, d, J=4.8 Hz), 5.74 (1H, dd, J=4.8 Hz, J=7.8 Hz), 6.71 (1H, d, J=6.6 Hz), 7.81 (1H, d, J=9.0 Hz), 8.02 (1H, s), 8.52 (1H, d, J=9.0 Hz), 8.60 (1H, d, J=6.6 Hz), 9.33 (1H, d, J=7.8 Hz), 9.58 (1H, broad s) 13.80 (1H, broad s). Anal. calcd. for C₂₁H₁₉ClN₄O₆S.HCl.1.5H₂O: C, 45.49; N, 10.11; found: C, 45.43; N, 10.05.

### Example 7

### Preparation of an Aminoquinoline-Cephalosporin Hybrid, ref PA 1088

### (6R,7R)-3-Acetoxymethyl-7-[2-(7-chloro-quinolin-4-ylamino)-acetylamino]-5,8-dioxo-5λ⁴-thia-1-aza-bicyclo[4.2.0]oct-2-ene-2-carboxylic acid hydrochloride

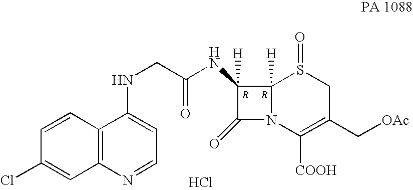

1.2 mL of anisole (10.7 mmol), followed by 2.0 mL of trifluoroacetic acid, injected dropwise (27.0 mmol), is added to a solution, at 0° C. under argon, of (6R,7R)-3-acetoxymethyl-7-[2-(7-chloro-quinolin-4-ylamino)-acetylamino]-5,8-dioxo-5×4-thia-1-aza-bicyclo[4.2.0]oct-2-ene-2-carboxylic acid benzhydryl ester (Example 5.3) (1.8 g, 2.7 mmol) in 20 mL of dry dichloromethane. The mixture is left under magnetic stirring for 1 hour and 30 minutes at room temperature. The product, as triflate salt, is precipitated by adding diethyl ether, filtered and washed with dichloromethane. The resulting solid is suspended in 20 mL of a chloroform/ethanol 1/1 v/v mixture and cooled to 0° C. Successively, 1.4 mL of 2M NH₃ in 2-propanol (2.7 mmol) left under stirring for 15 minutes and then 1.1 mL of 5N HCl in 2-propanol (4.0 mmol) left under stirring for 30 minutes are added to the suspension. The product is then precipitated in diethyl ether. The precipitate is filtered off, washed with chloroform, with ethanol, then with diethyl ether and dried under vacuum. PA 1088 is obtained as a slightly yellow powder (0.5 g, 24%).

¹H NMR (400 MHz, DMSO) δ ppm: 2.03 (3H, s), 3.62 (1H, d, J=18.4 Hz), 3.89 (1H, d, J=18.4 Hz), 4.41 (2H, m), 4.61 (1H, d, J=12.9 Hz), 4.92 (1H, d, J=4.0 Hz), 5.20 (1H, d, J=12.9 Hz), 5.89 (1H, dd, J=4.0 Hz, J=8.2 Hz), 6.73 (1H, d, 1=6.5 Hz), 7.79 (1H, d, J=9.0 Hz), 8.08 (1H, s), 8.55 (1H, d, J=9.0 Hz), 8.60 (1H, d, J=6.5 Hz), 8.65 (1H, d, J=8.2 Hz), 9.56 (1H, broad s), 13.76 (broad s). MS (IS>0) m/z: 507.2 (M−Cl)⁺. Anal. calcd. for C₂₁H₁₉ClN₄O₇S.HCl.2H₂O: C, 43.53; N, 9.67; found: C, 43.51; N, 9.59.

### Example 8

### Preparation of an Aminoquinoline-Cephalosporin Hybrid, ref PA 1092

### (6R,7R)-3-Acetoxymethyl-7-[2-(7-chloro-quinolin-4-ylamino)-acetylamino]-5,8-dioxo-5λ⁴-thia-1-aza-bicyclo[4.2.0]oct-2-ene-2-carboxylic acid

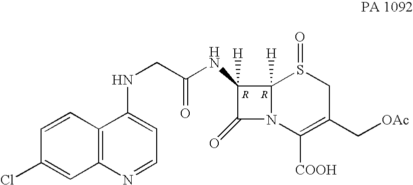

A suspension of PA 1088 (Example 7) (0.5 g, 0.8 mmol) is stirred for 30 minutes in 40 mL of water at room temperature. The product is filtered, washed with ethanol, then with diethyl ether and dried under vacuum. PA 1092 is obtained as a slightly yellow powder (0.2 g, 31%).

¹H NMR (250 MHz, DMSO) δ ppm: 2.00 (3H, s), 3.55 (1H, d, J=18.2 Hz), 3.85 (1H, d, J=18.2 Hz), 4.20 (2H, m), 4.57 (1H, d, J=12.5 Hz), 4.88 (1H, broad s), 5.18 (1H, d, J=12.5 Hz), 5.89 (1H, broad s), 6.51 (1H, broad s), 7.60 (1H, d, J=9.0 Hz), 7.88 (1H, s), 8.29-8.50 (4H, m). Anal. clacd. for C₂₁H₁₉ClN₄O₇S.3.5H₂O: C, 44.25; N, 9.83; found C, 44.21; N, 9.57.

### Example 9

### Preparation of an Aminoquinoline-Cephalosporin Hybrid, ref PA 1037

### (6R,7R)-3-Acetoxymethyl-7-[3-(7-chloro-quinolin-4-ylamino)-propionylamino]-8-oxo-5-thia-1-aza-bicyclo[4.2.0]oct-2-ene-2-carboxylic acid

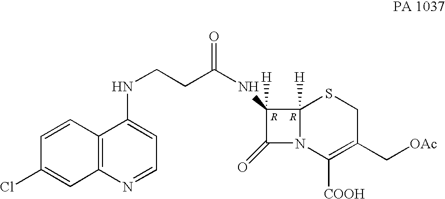

### 9.1. Mixture of (6R,7R)-3-acetoxymethyl-7-[3-(7-chloro-quinolin-4-ylamino)-propionyl-amino]-8-oxo-5-thia-1-aza-bicyclo[4.2.0]oct-2-ene-2-carboxylic acid benzhydryl ester and (6R,7R)-3-Acetoxymethyl-7-[3-(7-chloro-quinolin-4-ylamino)-propionyl-amino]-8-oxo-5-thia-1-aza-bicyclo[4.2.0]oct-3-ene-2-carboxylic acid benzhydryl ester: Δ²/Δ³

The coupling product is prepared according to the procedure described in Example 5.2 from 5.7 g of 3-(7-chloro-quinolin-4-ylamino)-propionic acid (Example 4.1) (19.8 mmol), 2.8 g of HOBT (20.7 mmol), 4.3 g of DCC (20.7 mmol), 8.7 g of (6R,7R)-3-acetoxymethyl-7-amino-8-oxo-5-thia-1-aza-bicyclo[4.2.0]oct-2-ene-2-carboxylic acid benzhydryl ester p-toluene sulfonic acid (Example 5.1) (19.8 mmol) and 2.8 mL of triethylamine (19.8 mmol). The coupling product is obtained after purification by liquid chromatography on silica gel (SiO₂ 60A C.C 70-200 μm, eluent: ethyl acetate/ethanol 90/10 v/v in order to get rid of the impurities and then ethyl acetate/ethanol/triethylamine 90/5/5 v/v/v), as an orangey powder (6.1 g, 46%) as a Δ²/Δ³ 20/80 mixture, used as such in the following step.

### 9.2. (6R,7R)-3-Acetoxymethyl-7-[3-(7-chloro-quinolin-4-ylamino)-propionylamino]-5,8-dioxo-5×4-thia-1-aza-bicyclo[4.2.0]oct-2-ene-2-carboxylic acid benzhydryl ester

The oxidation is carried out according to the procedure described in Example 5.3 from 6.4 g of the Δ²/Δ³ mixture of Example 9.1 (9.5 mmol) and 3.3 g of 3-chloroperoxybenzoic acid (19.0 mmol). The product is obtained as a yellow powder (4.9 g, 75%).

IR (KBr) cm⁻¹: (C═O) 1788, 1733, 1647. ¹H NMR (400 MHz, DMSO) δ ppm: 1.98 (3H, s), 2.71 (2H, t, J=6.9 Hz), 3.53 (2H, q, J=6.9 Hz), 3.65 (1H, d, J=18.7 Hz), 3.96 (1H, d, J=18.7 Hz), 4.62 (1H, d, J=13.4 Hz), 4.97 (1H, d, J=4.8 Hz), 5.08 (1H, d, J=13.4 Hz), 5.98 (1H, dd, J=4.8 Hz, J=8.2 Hz), 6.55 (1H, d, J=5.5 Hz), 6.96 (1H, s), 7.26-7.46 (9H, m), 7.47 (1H, dd, J=2.2 Hz, J=9.0 Hz), 7.53 (2H, d, J=7.3 Hz), 7.80 (1H, d, J=2.2 Hz), 8.25 (1H, d, J=9.0 Hz), 8.43 (1H, d, J=5.5 Hz), 8.50 (1H, d, J=8.2 Hz). MS (IS>0) m/z: 687.3 (M+H⁺).

### 9.3. (6R,7R)-3-Acetoxymethyl-7-[3-(7-chloro-quinolin-4-ylamino)-propionylamino]-8-oxo-5-thia-1-aza-bicyclo[4.2.0]oct-2-ene-2-carboxylic acid benzhydryl ester

The reduction is carried out according to the procedure described in Example 5.4 from 5.6 g of (6R,7R)-3-acetoxymethyl-7-[3-(7-chloro-quinolin-4-ylamino)-propionyl-amino]-5,8-dioxo-5λ⁴-thia-1-aza-bicyclo[4.2.0]oct-2-ene-2-carboxylic acid benzhydryl ester (Example 9.2) (8.2 mmol) and 1.6 mL of trichlorophosphine (18.3 mmol). The product is obtained after recryztallisation from dichloromethane/diethyl ether as a beige powder (5.0 g, 91%).

IR (KBr) cm⁻¹: (C═O) 1783, 1738, 1679. ¹H NMR (400 MHz, DMSO) δ ppm: 1.96 (3H, s), 2.72 (2H, t, J=6.8 Hz), 3.48 (1H, d, J=18.3 Hz), 3.64 (1H, d, J=18.3 Hz), 3.78 (2H, q, J=6.8 Hz), 4.62 (1H, d, J=13.0 Hz), 4.85 (1H, d, J=13.0 Hz), 5.15 (1H, d, J=4.9 Hz), 5.81 (1H, dd, J=4.9 Hz, J=8.3 Hz), 6.92 (1H, d, J=7.2 Hz), 6.92 (1H, s), 7.29-7.49 (10H, m), 7.79 (1H, dd, J=2.1 Hz, J=9.1 Hz), 8.07 (1H, d, J=2.1 Hz), 8.58 (1H, d, J=7.2 Hz), 8.62 (1H, d, J=9.1 Hz), 9.10 (1H, d, J=8.3 Hz), 9.54 (1H, t, J=6.8 Hz). MS (IS>0) m/z: 671.2 (M+H⁺).

### 9.4. (6R,7R)-3-Acetoxymethyl-7-[3-(7-chloro-quinolin-4-ylamino)-propionylamino]-8-oxo-5-thia-1-aza-bicyclo[4.2.0]oct-2-ene-2-carboxylic acid

The deprotection is carried out according to the procedure described in Example 5.5 from 3.0 g of (6R,7R)-3-acetoxymethyl-7-[3-(7-chloro-quinolin-4-ylamino)-propionyl-amino]-8-oxo-5-thia-1-aza-bicyclo [4.2.0]oct-2-ene-2-carboxylic acid benzhydryl ester (Example 9.3) (4.5 mmol), 2.0 mL of anisole (18.4 mmol) and 3.3 mL of trifluoroacetic acid (45.0 mmol). PA 1037 is obtained as an ivory powder after recrystallization by dissolution in 5% (w/v) aqueous Na₂CO₃ and precipitation at 0° C. with 2 M aqueous HCl until pH 6 (0.6 g, 27%).

IR (KBr) cm⁻¹: (C═O) 1773, 1753, 1653. ¹H NMR (400 MHz, DMSO) δ ppm: 2.02 (3H, s), 2.68 (2H, t, J=6.7 Hz), 3.39 (1H, d, J=18.0 Hz), 3.58 (1H, d, J=18.0 Hz), 3.71 (2H, m), 4.68 (1H, d, J=12.7 Hz), 5.00 (1H, d, J=12.7 Hz), 5.07 (1H, d, J=4.9 Hz), 5.70 (1H, dd, J=4.9 Hz, J=8.2 Hz), 6.83 (1H, d, J=6.8 Hz), 7.71 (1H, dd, J=2.1 Hz, J=9.1 Hz), 7.96 (1H, d, J=2.1 Hz), 8.46 (1H, d, J=9.1 Hz), 8.54 (1H, d, J=6.8 Hz), 8.94 (1H, broad s), 9.06 (1H, d, J=8.2 Hz). MS (IS>0) m/z: 505.0 (M+H⁺). Anal. calcd. for C₂₂H₂₁ClN₄O₆S.3H₂O: C, 47.27; N, 10.02; found: C, 47.34; N, 9.93.

### Example 10

### Preparation of an Aminoquinoline-Cephalosporin Hybrid, ref PA 1063

### (6R,7R)-3-Acetoxymethyl-7-[3-(7-chloro-quinolin-4-ylamino)-propionyl-amino]-5,8-dioxo-5X⁴-thia-1-aza-bicyclo[4.2.0]oct-2-ene-2-carboxylic acid

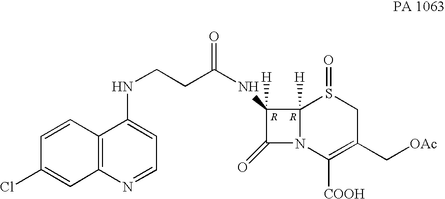

The deprotection is carried out according to the procedure described in Example 5.5 from 1.1 g of (6R,7R)-3-acetoxymethyl-7-[3-(7-chloro-quinolin-4-ylamino)-propionyl-amino]-5,8-dioxo-5λ⁴-thia-1-aza-bicyclo[4.2.0]oct-2-ene-2-carboxylic acid benzhydryl ester (Example 9.2) (1.6 mmol), 0.7 mL of anisole (6.2 mmol) and 1.2 mL of trifluoroacetic acid (15.5 mmol). PA 1063 is obtained as an ivory powder (0.6 g, 27%).

IR (KBr) cm⁻¹: (C═O) 1774, 1732, 1647. ¹H NMR (250 MHz, DMSO) δ ppm: 2.01 (3H, s), 2.71 (2H, broad s), 3.55 (1H, d, J=18.6 Hz), 3.59 (2H, broad s), 3.78 (1H, d, J=18.6 Hz), 4.59 (1H, d, J=12.9 Hz), 4.85 (1H, broad s), 5.21 (1H, d, J=12.9 Hz), 5.79 (1H, broad s), 6.67 (1H, broad s), 7.58 (1H, d, J=9.0 Hz), 7.87 (1H, s), 8.11 (1H, broad s), 8.33-8.46 (3H, m). MS (IS>0) m/z: 521.1 (M+H⁺).

### Example 11

### Preparation of an Aminoquinoline-Cephalosporin Hybrid, ref PA 1082

### (6R,7R)-3-Acetoxymethyl-7-[3-(7-chloro-quinolin-4-ylamino)-propionylamino]-5,8-dioxo-5×4-thia-1-aza-bicyclo[4.2.0]oct-2-ene-2-carboxylic acid hydrochloride

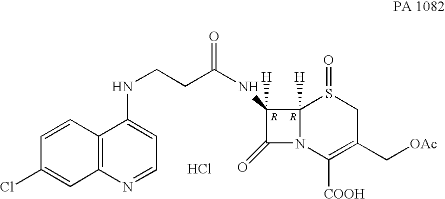

PA 1082 is prepared according to the procedure described in Example 6 from 0.7 g of PA 1063 (Example 10) (1.4 mmol) and 0.5 mL of 5M HCl in 2-propanol (4.0 mmol). PA 1082 is obtained as an ivory powder (0.4 g, 55%).

¹H NMR (250 MHz, DMSO) 8 ppm: 2.02 (3H, s), 2.76 (2H, m) 3.60 (1H, d, =18.6 Hz), 3.76 (2H, m), 3.85 (1H, d, J=18.6 Hz), 4.58 (1H, d, J=13.1 Hz), 4.89 (1H, d, J=4.2 Hz), 5.20 (1H, d, J=13.1 Hz), 5.83 (1H, dd, J=4.2 Hz, J=7.7 Hz), 6.91 (1H, d, J=7.1 Hz), 7.80 (1H, d, J=8.8 Hz), 8.04 (1H, s), 8.55 (3H, m), 9.43 (1H, broad s), 14.04 (broad s). MS (IS>0) m/z: 521.1 (M−Cl)⁺. Anal. calcd. for C₂₂H₂₁ClN₄O₇S.HCl.1.5H₂O.0.1Et₂O: C, 44.77; N, 9.32; found: C, 44.83; N, 9.25.

### Example 12

### Preparation of an Aminoquinoline-Cephalosporin Hybrid, ref PA 1053

### (6R,7R)-3-Acetoxymethyl-7-[4-(7-chloro-quinolin-4-ylamino)-butyrylamino]-8-oxo-5-thia-1-aza-bicyclo[4.2.0]oct-2-ene-2-carboxylic acid

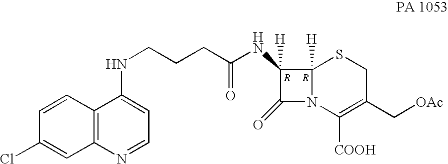

### 12.1. 4-(7-Chloro-quinolin-4-yl)-butyric acid

This compound is prepared according to the procedure described in Example 1.1 from 30.0 g of 4,7-dichloroquinoline (0.15 mol), 32.8 g of 4-aminobutyric acid (0.32 mol) and 77.0 g of phenol (0.82 mol). The product is obtained as a white powder (32.7 g, 82%).

¹H NMR (300 MHz, CF₃COOD) δ ppm: 2.23 (2H, quint, J=6.9 Hz), 2.71 (2H, t, J=6.9 Hz), 3.71 (2H, t, J=6.9 Hz), 6.81 (1H, d, J=7.5 Hz), 7.64 (1H, dd, J=1.8 Hz, J=9.0 Hz), 7.82 (1H, d, J=1.8 Hz), 8.08 (1H, d, J=9.0 Hz), 8.22 (1H, d, J=7.5 Hz).

### 12.2. Mixture of (6R,7R)-3-acetoxymethyl-7-[4-(7-chloro-quinolin-4-ylamino)-butyrylamino]-8-oxo-5-thia-1-aza-bicyclo[4.2.0]oct-2-ene-2-carboxylic acid benzhydryl ester and (6R,7R)-3-acetoxymethyl-7-[4-(7-chloro-quinolin-4-ylamino)-butyrylamino]-8-oxo-5-thia-1-aza-bicyclo [4.2.0]oct-3-ene-2-carboxylic acid benzhydryl ester: Δ²/Δ³

The coupling product is prepared according to the procedure described in Example 5.2 from 7.8 g of 4-(7-chloro-quinolin-4-yl)-butyric acid (Example 12.1) (24.5 mmol), 3.5 g of HOBT (25.7 mmol), 5.3 g of DCC (25.7 mmol), 15.1 g of (6R,7R)-3-acetoxymethyl-7-amino-8-oxo-5-thia-1-aza-bicyclo[4.2.0]oct-2-ene-2-carboxylic acid benzhydryl ester p-toluenesulfonic acid (Example 5.1) (24.5 mmol) and 6.9 mL of triethylamine (49.5 mmol). The coupling product is obtained after purification by liquid chromatography on silica gel (SiO₂ 60A C.C 70-200 μm, eluent: ethyl acetate/ethanol/triethylamine 90/9/1 v/v/v) as an orangey powder (3.3 g, 20%) as a Δ²/Δ³ 22/78 mixture, used as such in the following step.

### 12.3. (6R,7R)-3-Acetoxymethyl-7-[4-(7-chloro-quinolin-4-ylamino)-butyrylamino]-5,8-dioxo-5×4-thia-1-aza-bicyclo[4.2.0]oct-2-ene-2-carboxylic acid benzhydryl ester

The oxidation is carried out according to the procedure described in Example 5.3 from 3.3 g of the Δ²/Δ³ mixture of Example 12.2 (4.8 mmol) and 1.7 g of 3-chloroperoxybenzoic acid (9.6 mmol). The product is obtained as an orange-yellow powder (2.5 g, 74%).

IR (KBr) cm⁻¹: (C═O) 1791, 1735, 1655. ¹H NMR (300 MHz, DMSO) δ ppm: 1.92 (2H, quint, J=7.2 Hz), 2.01 (3H, s), 2.43 (2H, t, J=7.2 Hz), 3.33 (2H, m), 3.65 (1H, d, J=18.9 Hz), 3.96 (1H, d, J=18.9 Hz), 4.61 (1H, d, =13.2 Hz), 4.96 (1H, d, J=4.2 Hz), 5.07 (1H, d, J=13.2 Hz), 5.95 (1H, dd, =4.2 Hz, J=7.8 Hz), 6.57 (1H, d, J=5.7 Hz), 6.95 (1H, s), 7.27-7.54 (11H, m), 7.66 (1H, broad s), 7.80 (1H, d, J=2.1 Hz), 8.30 (1H, d, J=9.0 Hz), 8.32 (1H, d, J=7.8 Hz), 8.43 (1H, d, J=5.7 Hz). MS (IS>0) m/z: 701.3 (M+H⁺).

### 12.4. (6R,7R)-3-Acetoxymethyl-7-[4-(7-chloro-quinolin-4-ylamino)-butyrylamino]-8-oxo-5-thia-1-aza-bicyclo[4.2.0]oct-2-ene-2-carboxylic acid benzhydryl ester

The reduction is carried out according to the procedure described in Example 5.4 from 2.9 g of (6R,7R)-3-acetoxymethyl-7-[4-(7-chloro-quinolin-4-ylamino)-butyrylamino]-5,8-dioxo-5λ⁴-thia-1-aza-bicyclo [4.2.0]oct-2-ene-2-carboxylic acid benzhydryl ester (Example 12.3) (4.1 mmol) and 0.8 mL of trichlorophosphine (9.1 mmol). The product is obtained after recrystallization from dichloromethane/diethyl ether as a beige powder (2.5 g, 89%).

IR (KBr) cm⁻¹: (C═O) 1784, 1730, 1661. ¹H NMR (300 MHz, DMSO) δ ppm: 1.92 (2H, quint, J=7.2 Hz), 1.96 (3H, s), 2.38 (2H, t, J=7.2 Hz), 3.53 (2H, m), 3.53 (1H, d, J=18.6 Hz), 3.67 (1H, d, =18.6 Hz), 4.62 (1H, d, J=12.9 Hz), 4.86 (1H, d, J=12.9 Hz), 5.16 (1H, d, =4.8 Hz), 5.79 (1H, dd, J=4.8 Hz, J=8.1 Hz), 6.91 (1H, d, J=6.9 Hz), 6.92 (1H, s), 7.28-7.49 (10H, m), 7.78 (1H, dd, 1=1.8 Hz, J=9.0 Hz), 8.04 (1H, d, J=1.8 Hz), 8.56 (1H, d, J=6.9 Hz), 8.63 (1H, d, J=9.0 Hz), 8.97 (1H, d, J=8.1 Hz), 9.56 (1H, broad s). MS (IS>0) m/z: 685.2 (M+H⁺).

### 12.5. (6R,7R)-3-Acetoxymethyl-7-[4-(7-chloro-quinolin-4-ylamino)-butyrylamino]-8-oxo-5-thia-1-aza-bicyclo[4.2.0]oct-2-ene-2-carboxylic acid

The deprotection is carried out according to the procedure described in Example 5.5 from 0.8 g of (6R,7R)-3-acetoxymethyl-7-[4-(7-chloro-quinolin-4-ylamino)-butyrylamino]-8-oxo-5-thia-1-aza-bicyclo[4.2.0] oct-2-ene-2-carboxylic acid benzhydryl ester (Example 12.4) (1.2 mmol), 0.5 mL of anisole (4.8 mmol) and 0.9 mL of trifluoroacetic acid (12.1 mmol). PA 1053 is obtained as a white powder after recrystallization by dissolution in 5% (w/v) aqueous Na₂CO₃ and precipitation at 0° C. with 2 M aqueous HCl until pH 6 (0.3 g, 35%).

IR (KBr) cm⁻¹: (C═O) 1769, 1737, 1653. ¹H NMR (300 MHz, DMSO) δ ppm: 1.91 (2H, m), 2.02 (3H, s), 2.37 (2H, t, J=7.2 Hz), 3.41 (2H, m), 3.44 (1H, d, J=18.3 Hz), 3.61 (1H, d, J=18.3 Hz), 4.69 (1H, d, J=12.9 Hz), 5.00 (1H, d, J=12.9 Hz), 5.09 (1H, d, J=4.8 Hz), 5.68 (1H, dd, J=4.8 Hz, J=8.1 Hz), 6.73 (1H, d, J=6.0 Hz), 7.64 (1H, d, J=9.0 Hz), 7.89 (1H, s), 8.41 (1H, d, J=9.0 Hz), 8.52 (2H, broad m), 8.90 (1H, d, J=6.0 Hz). MS (IS>0) m/z: 519.2 (M+H⁺). Anal. calcd. for C₂₃H₂₃ClN₄O₆S.2H₂O: C, 49.77; N, 10.10; found: C, 49.79; N, 9.74.

### Example 13

### Preparation of an Aminoquinoline-Cephalosporin Hybrid, ref PA 1054

### (6R,7R)-3-Acetoxymethyl-7-{[1-(7-chloro-quinolin-4-yl)-piperidine-4-carbonyl]-amino}-8-oxo-5-thia-1-aza-bicyclo[4.2.0]oct-2-ene-2-carboxylic acid

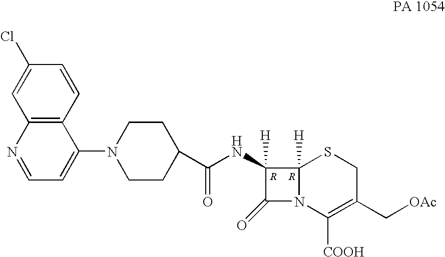

### 13.1. Mixture of (6R,7R)-3-acetoxymethyl-7-{[1-(7-chloro-quinolin-4-yl)-piperidine-4-carbonyl]-amino}-8-oxo-5-thia-1-aza-bicyclo[4.2.0]oct-2-ene-2-carboxylic acid benzhydryl ester and (6R,7R)-3-acetoxymethyl-7-{[1-(7-chloro-quinolin-4-yl)-piperidine-4-carbonyl]-amino}-8-oxo-5-thia-1-aza-bicyclo[4.2.0]oct-3-ene-2-carboxylic acid benzhydryl ester: Δ²/Δ³

2.1 mL of N-methylmorpholine (19.4 mmol) are added to a mixture of 1-(7-chloro-quinolin-4-yl)-piperidine-4-carboxylic acid (Example 1.1) (1.2 g, 3.9 mmol) and (6R,7R)-3-acetoxymethyl-7-amino-8-oxo-5-thia-1-aza-bicyclo[4.2.0]oct-2-ene-2-carboxylic acid benzhydryl ester p-toluene sulfonic acid (Example 5.1) (2.4 g, 3.9 mmol) in 40 mL of DMF. The suspension is left under magnetic stirring for 15 minutes before adding the activating agent PyBOP® (2.0 g, 3.9 mmol). After stirring for additional 24 hours at room temperature, the reaction mixture is diluted with 50 mL of dichloromethane and then washed successively with 50 mL of 5% (w/v) aqueous Na₂CO₃, twice 50 mL of water and 50 mL of water saturated with NaCl. The organic phase is dried over magnesium sulfate, filtered and then evaporated. The coupling product is obtained as an orangey powder (2.5 g, 90%) as a Δ²/Δ³ 32/68 mixture, used as such in the following step.

### 13.2. (6R,7R)-3-Acetoxymethyl-7-{[1-(7-chloro-quinolin-4-yl)-piperidine-4-carbonyl]-amino}-5,8-dioxo-5×4-thia-1-aza-bicyclo[4.2.0]oct-2-ene-2-carboxylic acid benzhydryl ester

A solution of 3-chloroperoxybenzoic acid (4.9 g, 28.4 mmol) in 100 mL of dichloromethane is added dropwise, over a period of 3 hours, to a solution, at 0° C., of the Δ²/Δ³ mixture of Example 13.1 (10.1 g, 14.2 mmol) in 100 mL of dichloromethane. The reaction mixture is then washed with a mixture of 100 mL of 5% (w/v) aqueous NaHCO₃ and 100 ml of 5% (w/v) aqueous sodium sulfite. The organic phase is dried over magnesium sulfate, filtered and then evaporated. The product is purified by liquid chromatography on silica gel (SiO₂ 60A C.C 70-200 μm, eluent: dichloromethane/ethanol 90/10 v/v). The cleanest fractions on TLC revealed under UV are evaporated. The product is obtained as an ivory powder (4.0 g, 38%).

IR (KBr) cm⁻¹: (C═O) 1787, 1733, 1653. ¹H NMR (400 MHz, CDCl₃) δ ppm: 2.05 (3H, s), 2.10 (4H, m), 2.50 (1H, m), 2.87 (2H, m), 3.28 (1H, d, J=19.2 Hz), 3.88 (1H, d, J=19.2 Hz), 3.63 (2H, d, J=12.0 Hz), 4.56 (1H, d, J=4.8 Hz), 4.78 (1H, d, J=14.4 Hz), 5.32 (1H, d, J=14.4 Hz), 6.18 (1H, dd, J=4.8 Hz, J=9.6 Hz), 6.83 (1H, d, J=5.2 Hz), 6.97 (1H, s), 6.97 (1H, d, J=9.6 Hz), 7.27-7.49 (11H, m), 7.92 (1H, d, J=9.2 Hz), 8.05 (1H, d, J=2.0 Hz), 8.71 (1H, d, J=5.2 Hz). MS (IS>0) m/z: 727.3 (M+H⁺).

### 13.3. (6R,7R)-3-Acetoxymethyl-7-{[1-(7-chloro-quinolin-4-yl)-piperidine-4-carbonyl]-amino}-8-oxo-5-thia-1-aza-bicyclo[4.2.0]oct-2-ene-2-carboxylic acid benzhydryl ester

0.2 mL of trichlorophosphine (1.9 mmol) is added dropwise to a solution, at −20° C. under argon, of (6R,7R)-3-acetoxymethyl-7-{[1-(7-chloro-quinolin-4-yl)-piperidine-4-carbonyl]-amino}-5,8-dioxo-5λ⁴-thia-1-aza-bicyclo[4.2.0]oct-2-ene-2-carboxylic acid benzhydryl ester (Example 13.2) (0.6 g, 0.9 mmol) in 6 mL of dry DMF. The reaction is stirred for 1 hour at −20° C. The mixture is then diluted with 20 mL of dichloromethane and washed successively with twice 20 mL of water and 20 mL of water saturated with NaCl. The organic phase is dried over magnesium sulfate, filtered and then evaporated. The product is obtained after recryztallisation from dichloromethane/diethyl ether as an ivory powder (0.5 g, 83%).

IR (KBr) cm⁻¹: (C═O) 1783, 1732, 1672. ¹H NMR (400 MHz, CDCl₃) δ ppm: 2.02 (3H, s), 2.11-2.23 (4H, m), 3.07 (1H, m), 3.20 (1H, d, J=18.8 Hz), 3.45 (2H, m), 3.50 (1H, d, J=18.8 Hz), 4.02 (2H, m), 4.48 (1H, d, J=14.1 Hz), 4.95 (1H, d, J=14.1 Hz), 4.99 (1H, d, J=4.9 Hz), 5.94 (1H, dd, J=4.9 Hz, J=8.5 Hz), 6.48 (1H, d, J=6.7 Hz), 6.82 (1H, s), 7.30-7.55 (12H, m), 7.91 (1H, d, J=9.2 Hz), 8.29 (1H, d, J=8.5 Hz), 8.33 (1H, d, J=6.7 Hz), 8.48 (1H, d, J=1.8 Hz). SM (IS>0) m/z: 711.2 (M+H⁺).

### 13.4. (6R,7R)-3-Acetoxymethyl-7-{[1-(7-chloro-quinolin-4-yl)-piperidine-4-carbonyl]-amino}-8-oxo-5-thia-1-aza-bicyclo[4.2.0]oct-2-ene-2-carboxylic acid

0.3 mL of anisole (2.5 mmol), followed by 0.5 ml of trifluoroacetic acid injected dropwise (6.3 mmol), are added to a solution, at 0° C. under argon, of (6R,7R)-3-acetoxymethyl-7-{[1-(7-chloro-quinolin-4-yl)-piperidine-4-carbonyl]-amino}-8-oxo-5-thia-1-aza-bicyclo[4.2.0]oct-2-ene-2-carboxylic acid benzhydryl ester (0.5 g, 0.6 mmol) (Example 13.3) in 10 mL of dry dichloromethane. The reaction is stirres for 1 hour and 30 minutes at room temperature. The product, as a triflate salt, is precipitated by adding diethyl ether and filtered. The resulting solid is washed with water, acetone, diethyl ether before being dried under vacuum. PA 1054 is obtained as an off-white powder (0.2 g, 44%).

IR (KBr) cm⁻¹: (C═O) 1763, 1737, 1648. ¹H NMR (400 MHz, DMSO) 8 ppm: 1.86-1.98 (4H, m), 2.02 (3H, s), 2.58 (1H, m), 2.85 (2H, m), 3.30 (1H, d, J=17.2 Hz), 3.53 (1H, d, J=17.2 Hz), 3.56 (2H, m), 4.75 (1H, d, J=12.4 Hz), 5.01 (1H, d, J=12.4 Hz), 5.03 (1H, d, J=4.4 Hz), 6.03 (1H, dd, J=4.4 Hz, J=8.0 Hz), 7.02 (1H, d, J=5.0 Hz), 7.56 (1H, dd, J=2.0 Hz, J=9.2 Hz), 7.97 (1H, d, J=2.0 Hz), 8.01 (1H, d, J=9.2 Hz), 8.69 (1H, d, J=5.0 Hz), 8.89 (1H, d, J=8.0 Hz). MS (IS>0) m/z: 545.2 (M+H⁺).

### Example 14

### Preparation of an Aminoquinoline-Cephalosporin Hybrid, ref PA 1074

(6R,7R)-3-Acetoxymethyl-7-{[1-(7-chloro-quinolin-4-yl)-piperidine-4-carbonyl]-amino}-8-oxo-5-thia-1-aza-bicyclo[4.2.0]oct-2-ene-2-carboxylic acid hydrochloride

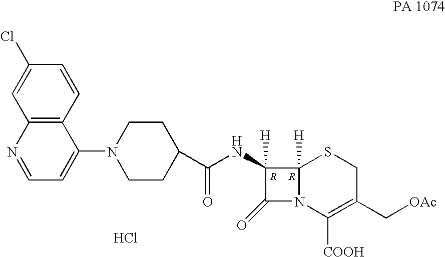

0.2 mL of 5M HCl in 2-propanol (1.0 mmol) is added dropwise to a solution, at 0° C., of PA 1054 (Example 13.4) (0.5 g, 0.8 mmol) in 40 mL of a mixture of chloroform/ethanol 1/1 v/v. After 20 minutes of stirring at 0° C., the product is precipitated in diethyl ether. The precipitate is filtered off, washed with cold acetone and then with diethyl ether and dried under vacuum. PA 1074 is obtained as an off-white powder (0.3 g, 54%).

IR (KBr) cm⁻¹: (C═O) 1779, 1736, 1668. ¹H NMR (300 MHz, DMSO) δ ppm: 1.79-1.99 (4H, m), 2.03 (3H, s), 2.74 (1H, m), 3.43 (2H, m), 3.55 (1H, d, J=18.3 Hz), 3.64 (1H, d, J=18.3 Hz), 4.12 (2H, d, J=12.6 Hz), 4.68 (1H, d, J=12.9 Hz), 5.00 (1H, d, J=12.9 Hz), 5.12 (1H, d, J=4.5 Hz), 5.69 (1H, dd, J=4.5 Hz, J=8.1 Hz), 7.20 (1H, d, J=7.2 Hz), 7.68 (1H, dd, J=1.5 Hz, J=9.0 Hz), 8.11 (1H, d, J=1.5 Hz), 8.15 (1H, d, J=9.0 Hz), 8.65 (1H, d, J=7.2 Hz), 8.98 (1H, d, J=8.1 Hz). MS (IS>0) m/z: 545.2 (M+H⁺). Anal. calcd. for C₂₅H₂₅ClN₄O₆S.HCl.2.5H₂O: C, 47.92; N, 8.94; found: C, 47.89; N, 8.92.

### Example 15

### Preparation of an Aminoquinoline-Cephalosporin Hybrid, ref PA 1100

### (6R,7R)-3-Acetoxymethyl-7-[2-(7-trifluoromethyl-quinolin-4-ylamino)-acetylamino]-8-oxo-5-thia-1-aza-bicyclo[4.2.0]oct-2-ene-2-carboxylic acid

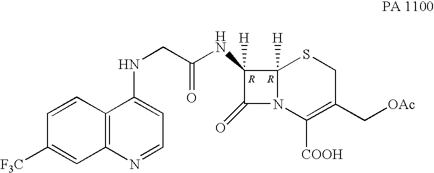

### 15.1. (7-Trifluoromethyl-quinolin-4-ylamino)-acetic acid

This compound is prepared according to the procedure described in Example 1.1 from a mixture of 2.5 g of 4-chloro-7-(trifluoromethyl)quinoline (10.8 mmol), 1.8 g of glycine (23.7 mmol) and 5.7 g of phenol (60.4 mmol) heated for 24 hours at 150° C. The product is obtained as a white powder (1.8 g, 62%).

¹H NMR (300 MHz, DMSO) 8 ppm: 4.10 (2H, d, J=6.0 Hz), 6.48 (1H, d, J=5.4 Hz), 7.72 (1H, dd, J=1.8 Hz, J=9.0 Hz), 7.83 (1H, t, J=6.0 Hz), 8.11 (1H, d, J=1.8 Hz), 8.43 (1H, d, J=9.0 Hz), 8.52 (1H, d, J=5.4 Hz).

### 15.2. Mixture of (6R,7R)-3-acetoxymethyl-7-[2-(7-trifluoromethyl-quinolin-4-ylamino)-acetylamino]-8-oxo-5-thia-1-aza-bicyclo[4.2.0]oct-2-ene-2-carboxylic acid benzhydryl ester and (6R,7R)-3-acetoxymethyl-7-[2-(7-trifluoromethyl-quinolin-4-ylamino)-acetylamino]-8-oxo-5-thia-1-aza-bicyclo[4.2.0]oct-3-ene-2-carboxylic acid benzhydryl ester: Δ²/Δ³

The coupling product is prepared according to the procedure described in Example 13.1 from 0.7 g of (7-trifluoromethyl-quinolin-4-ylamino)-acetic acid (Example 15.1) (2.6 mmol), 1.6 g of (6R,7R)-3-acetoxymethyl-7-amino-8-oxo-5-thia-1-aza-bicyclo[4.2.0]oct-2-ene-2-carboxylic acid benz-hydryl ester p-toluene sulfonic acid (Example 5.1) (2.6 mmol), 1.4 mL of N-methylmorpholine (13.0 mmol) and 1.3 g of PyBOP® (2.6 mmol). The coupling product is obtained after purification by liquid chromatography on silica gel (SiO₂ 60A C.C 6-35 μm, eluent: ethyl acetate/triethylamine/ethanol 96/3/1 v/v/v) as a clear beige powder (0.6 g, 32%) as a Δ²/Δ³ 31/69 mixture, used as such in the following step.

### 15.3. (6R,7R)-3-Acetoxymethyl-7-[2-(7-trifluoromethyl-quinolin-4-ylamino)-acetylamino]-5,8-dioxo-5λ⁴-thia-1-aza-bicyclo[4.2.0]oct-2-ene-2-carboxylic acid benzhydryl ester

The oxidation is carried out according to the procedure described in Example 5.3 from 0.6 g of the Δ²/Δ³ mixture of Example 15.2 (0.8 mmol) and 0.3 g of 3-chloroperoxybenzoic acid (1.7 mmol). The product is obtained as a yellow powder (0.5 g, 91%).

IR (KBr) cm⁻¹: (C═O) 1786, 1734, 1668. ¹H NMR (300 MHz, DMSO) δ ppm: 1.95 (3H, s), 3.60 (1H, d, J=18.6 Hz), 3.93 (1H, d, J=18.6 Hz), 4.15 (2H, m), 4.57 (1H, d, J=13.5 Hz), 4.95 (1H, d, J=4.8 Hz), 5.02 (1H, d, J=13.5 Hz), 6.04 (1H, dd, J=4.8 Hz, J=9.0 Hz), 6.51 (1H, d, J=5.4 Hz), 6.94 (1H, s), 7.26-7.52 (10H, m), 7.75 (1H, dd, J=1.5 Hz, J=8.7 Hz), 8.01 (1H, broad s), 8.13 (1H, d, J=1.5 Hz), 8.38 (1H, d, J=9.0 Hz), 8.40 (1H, d, J=8.7 Hz), 8.56 (1H, d, J=5.4 Hz). MS (IS>0) m/z: 707.2 (M+H⁺).

### 15.4. (6R,7R)-3-Acetoxymethyl-7-[2-(7-trifluoromethyl-quinolin-4-ylamino)-acetylamino]-8-oxo-5-thia-1-aza-bicyclo[4.2.0]oct-2-ene-2-carboxylic acid benzhydryl ester

The reduction is carried out according to the procedure described in Example 5.4 from 0.4 g of (6R,7R)-3-acetoxymethyl-7-[2-(7-trifluoromethyl-quinolin-4-ylamino)-acetylamino]-5,8-dioxo-5×4-thia-1-aza-bicyclo[4.2.0]oct-2-ene-2-carboxylic acid benzhydryl ester (Example 15.3) (0.6 mmol) and 0.1 mL of trichlorophosphine (1.3 mmol). The product is obtained after recrystallization from dichloromethane/diethyl ether as a beige powder (0.2 g, 54%).

¹H NMR (300 MHz, DMSO) δ ppm: 1.96 (3H, s), 3.35 (2H, m), 4.37 (2H, m), 4.64 (1H, d, J=12.9 Hz), 4.93 (1H, broad s), 4.96 (1H d, J=12.9 Hz), 5.78 (1H, broad s), 6.55 (1H, broad s), 6.87 (1H, s), 7.25-7.43 (10H, m), 7.62 (1H, broad s), 8.29 (1H, broad s), 8.39 (1H, broad s), 8.56 (1H, broad s), 8.93 (1H, broad s), 9.32 (1H, broad s).

### 15.5. (6R,7R)-3-Acetoxymethyl-7-[2-(7-trifluoromethyl-quinolin-4-ylamino)-acetylamino]-8-oxo-5-thia-1-aza-bicyclo[4.2.0]oct-2-ene-2-carboxylic acid

The deprotection is carried out according to the procedure described in Example 5.5 from 0.2 g of (6R,7R)-3-acetoxymethyl-7-[2-(7-trifluoromethyl-quinolin-4-ylamino)-acetylamino]-8-oxo-5-thia-1-aza-bicyclo[4.2.0]oct-2-ene-2-carboxylic acid benzhydryl ester (Example 15.4) (0.3 mmol), 0.1 mL of anisole (1.3 mmol) and 0.2 mL of trifluoroacetic acid (3.2 mmol). PA 1100 is obtained as a yellow powder after successive washings with water, acetonitrile and diethyl ether (0.1 g, 54%).

IR (KBr) cm⁻¹: (C═O) 1772, 1734, 1674. ¹H NMR (300 MHz, DMSO) 8 ppm: 2.03 (3H, s), 3.49 (1H, d, J=18.0 Hz), 3.63 (1H, d, J=18.0 Hz), 4.17 (2H, d, J=5.7 Hz), 4.69 (1H, d, J=12.8 Hz), 5.00 (1H, d, J=12.8 Hz), 5.11 (1H, d, J=4.8 Hz), 5.73 (1H, dd, J=4.8 Hz, J=8.4 Hz), 6.54 (1H, d, J=5.4 Hz), 7.83 (1H, d, J=9.0 Hz), 8.16 (1H, s), 8.43 (1H, broad s), 8.51 (1H, d, J=9.0 Hz), 8.58 (1H, broad s), 9.25 (1H, d, J=8.4 Hz). MS (IS>0) m/z: 525.3 (M+H⁺). Anal. calcd. for C₂₂H₁₉F₃N₄O₆S.3.5H₂O: C, 44.97; N, 9.54; found: C, 44.94; N, 9.15.

### Example 16

### Preparation of an Aminoquinoline-Cephalosporin Hybrid, ref PA 1101

### (6R,7R)-3-Acetoxymethyl-7-[2-(2-methyl-quinolin-4-ylamino)-acetylamino]-8-oxo-5-thia-1-aza-bicyclo[4.2.0]oct-2-ene-2-carboxylic acid

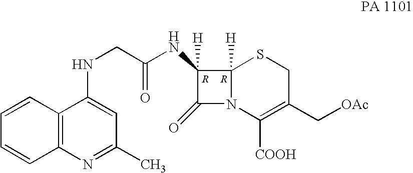

### 16.1. (2-Methyl-quinolin-4-ylamino)-acetic acid

This compound is prepared according to the procedure described in Example 1.1 from a mixture of 4.8 g of 4-chloroquinaldine (27.3 mmol), 4.5 g of glycine (60.0 mmol) and 14.6 g of phenol (155.0 mmol) heated for 24 hours at 150° C. The product is obtained as a white powder (3.8 g, 64%).

¹H NMR (300 MHz, CF₃COOD) δ ppm: 2.60 (3H, s), 4.37 (2H, s), 6.42 (1H, s), 7.59 (1H, t, J=7.2 Hz), 7.66 (1H, d, J=8.4 Hz), 7.80 (1H, t, J=7.5 Hz), 7.95 (1H, d, J=8.7 Hz).

### 16.2. Mixture of (6R,7R)-3-acetoxymethyl-7-[2-(2-methyl-quinolin-4-ylamino)-acetylamino]-8-oxo-5-thia-1-aza-bicyclo[4.2.0]oct-2-ene-2-carboxylic acid benzhydryl ester and (6R,7R)-3-acetoxymethyl-7-[2-(2-methyl-quinolin-4-ylamino)-acetylamino]-8-oxo-5-thia-1-aza-bicyclo[4.2.0]oct-3-ene-2-carboxylic acid benzhydryl ester: Δ²/Δ³

The coupling product is prepared according to the procedure described in Example 13.1 from 0.7 g of (2-methyl-quinolin-4-ylamino)-acetic acid (Example 16.1) (3.5 mmol), 2.2 g of (6R,7R)-3-acetoxymethyl-7-amino-8-oxo-5-thia-1-aza-bicyclo[4.2.0]oct-2-ene-2-carboxylic acid benzhydryl ester p-toluene sulfonic acid (Example 5.1) (3.5 mmol), 1.9 mL of N-methylmorpholine (17.5 mmol) and 1.8 g of PyBOP® (3.5 mmol). The coupling product is obtained after purification by liquid chromatography on silica gel (SiO₂ 60A C.C 6-35 μm, eluent: ethyl acetate/triethylamine/ethanol 95/3/2 v/v/v) as an orangey powder (1.3 g, 58%) as a Δ²/Δ³ 21/79 mixture, used as such in the following step.

### 16.3. (6R,7R)-3-Acetoxymethyl-7-[2-(2-methyl-quinolin-4-ylamino)-acetylamino]-5,8-dioxo-5×4-thia-1-aza-bicyclo[4.2.0]oct-2-ene-2-carboxylic acid benzhydryl ester

The oxidation is carried out according to the procedure described in Example 5.3 from 1.3 g of the Δ²/Δ³ mixture of Example 16.2 (2.0 mmol) and 0.7 g 3-chloroperoxybenzoic acid (4.0 mmol). The product is obtained as an orangey powder (1.1 g, 83%).

IR (KBr) cm⁻¹: (C═O) 1792, 1734, 1652. ¹H NMR (300 MHz, DMSO) 8 ppm: 1.95 (3H, s), 2.52 (3H, s), 3.62 (1H, d, J=18.9 Hz), 3.95 (1H, d, 3=18.9 Hz), 4.13 (2H, m), 4.58 (1H, d, 3=13.5 Hz), 4.96 (1H, d, J=4.2 Hz), 5.03 (1H, d, J=13.5 Hz), 6.04 (1H, dd, J=4.2 Hz, J=8.7 Hz), 6.34 (1H, s), 6.94 (1H, s), 7.26-7.54 (11H, m), 7.64 (1H, t, J=7.5 Hz), 7.75 (1H, d, J=7.8 Hz), 7.89 (1H, m), 8.17 (1H, d, J=8.7 Hz), 8.42 (1H, d, J=8.7 Hz). MS (IS>0) m/z: 653.2 (M+H⁺).

### 16.4. (6R,7R)-3-Acetoxymethyl-7-[2-(2-methyl-quinolin-4-ylamino)-acetylamino]-8-oxo-5-thia-1-aza-bicyclo[4.2.0]oct-2-ene-2-carboxylic acid benzhydryl ester

The reduction is carried out according to the procedure described in Example 5.4 from 2.3 g of (6R,7R)-3-acetoxymethyl-7-[2-(2-methyl-quinolin-4-ylamino)-acetylamino]-5,8-dioxo-5×4-thia-1-aza-bicyclo [4.2.0]oct-2-ene-2-carboxylic acid benzhydryl ester (Example 16.3) (3.5 mmol) and 0.7 mL of trichlorophosphine (7.8 mmol). The product is obtained after recrystallization from dichloromethane/diethyl ether as a beige powder (1.8 g, 82%).

IR (KBr) cm⁻¹: (C═O) 1784, 1733, 1639. ¹H NMR (300 MHz, DMSO) δ ppm: 1.96 (3H, s), 2.73 (3H, s), 3.58 (1H, d, J=18.3 Hz), 3.70 (1H, d, J=18.3 Hz), 4.33 (2H, m), 4.63 (1H, d, J=12.9 Hz), 4.87 (1H, d, J=12.9 Hz), 5.19 (1H, d, 7=5.1 Hz), 5.85 (1H, dd, J=5.1 Hz, J=8.1 Hz), 6.61 (1H, s), 6.93 (1H, s), 7.28-7.50 (10H, m), 7.70 (1H, m), 7.94 (2H, m), 8.46 (1H, d, J=8.7 Hz), 9.37 (1H, d, 1=8.1 Hz), 9.42 (1H, t, d, J=6.0 Hz). MS (IS>0) m/z: 637.2 (M+H⁺).

### 16.5. (6R,7R)-3-Acetoxymethyl-7-[2-(2-methyl-quinolin-4-ylamino)-acetylamino]-8-oxo-5-thia-1-aza-bicyclo[4.2.0]oct-2-ene-2-carboxylic acid

The deprotection is carried out according to the procedure described in Example 5.5 from 0.8 g of (6R,7R)-3-acetoxymethyl-7-[2-(2-methyl-quinolin-4-ylamino)-acetylamino]-8-oxo-5-thia-1-aza-bicyclo[4.2.0]oct-2-ene-2-carboxylic acid benzhydryl ester (Example 16.4) (1.2 mmol), 0.5 mL of anisole (4.9 mmol) and 0.9 mL of trifluoroacetic acid (12.4 mmol). PA 1101 is obtained as a yellow powder after successive washings with water, acetone and diethyl ether (0.2 g, 27%).

IR (KBr) cm⁻¹: (C═O) 1772, 1736, 1652. ¹H NMR (400 MHz, DMSO) δ ppm: 2.01 (3H, s), 2.59 (3H, s), 3.38 (1H, d, J=17.6 Hz), 3.58 (1H, d, J=17.6 Hz), 4.21 (2H, m), 4.74 (1H, d, J=12.4 Hz), 5.01 (1H, d, J=12.4 Hz), 5.06 (1H, d, J=4.8 Hz), 5.64 (1H, dd, J=4.8 Hz, J=8.0 Hz), 6.46 (1H, s), 7.58 (1H, t, J=7.4 Hz), 7.80 (1H, t, J=7.5 Hz), 7.89 (1H, d large, J=7.8 Hz), 8.33 (1H, d, J=8.6 Hz), 8.77 (1H, broad s), 9.26 (1H, d, J=8.0 Hz). MS (IS>0) m/z: 471.2 (M+H⁺). Anal. calcd. for C₂₂H₂₂N₄O₆S.2.5H₂O: C, 51.25; N, 10.87; found: C, 51.00; N, 10.79.

Example 17 below exemplifies the preparation of hybrid molecules of the family of aminoquinolines-quinolones (quinoloquines).

### Example 17

### Preparation of an Aminoquinoline-Quinolone Hybrid, ref PA 1123

### 7-[4-(7-Chloro-quinolin-4-yl)-piperazin-1-yl]-1-cyclopropyl-6-fluoro-4-oxo-1,4-dihydro-quinoline-3-carboxylic acid

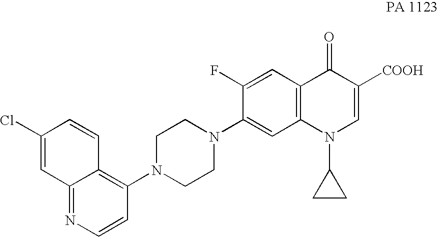

A mixture of 4,7-dichloroquinoline (0.3 g, 1.5 mmol), ciprofloxacin (0.5 g, 1.5 mmol) and phenol (0.7 g, 7.6 mmol) is heated at 140° C. under magnetic stirring for 24 hours. After cooling, the reaction mixture is diluted with 100 mL of dichloromethane. The resulting solid is filtered and then washed successively with water, methanol and diethyl ether before being dried under vacuum. PA 1123 is obtained as a yellow powder (0.5 g, 67%).

¹H NMR (300 MHz, DMSO) δ ppm: 1.23 (2H, m), 1.33 (2H, m), 3.72 (4H, m), 3.85 (1H, m), 4.04 (4H, m), 7.25 (1H, d, J=6.9 Hz), 7.59 (1H, d, J=7.5 Hz), 7.73 (1H, dd, J=2.1 Hz, J=9.3 Hz), 7.98 (1H, d, J=13.2 Hz), 8.11 (1H, d, J=2.1 Hz), 8.30 (1H, d, J=9.3 Hz), 8.69 (1H, s), 8.76 (1H, d, J=6.9 Hz). MS (IS>0) m/z: 493.2 (M+H⁺). Anal. calcd. for C₂₆H₂₂ClFN₄O₃.2H₂O: C, 59.03; N, 10.59; found: C, 58.67; N, 10.40.

Tests of Stability of the Hybrid Molecules and of Quantification of their Antibacterial Activity:

### Example 18

### Stability of the Hybrid Molecules at Physiological pH and at Acidic pH

The stability of the hybrid molecules given as Examples was determined, in solution at 37° C., at physiological pH (pH 7, phosphate buffer/acetonitrile, 75/25 v/v) and at acidic pH (pH 1, 0.1M HCl/ethanol, 70/30 v/v) by high pressure liquid chromatography coupled to a UV-visible detector (Beckman Coulter ODS C18 column, 5 μm, 4.6×250 mm; eluents: A: 0.1% TFA, B: CH₃CN/H₂O 90/10 0.1% TFA, gradient: from 10% to 100% of B in 30 minutes, and then 100% of B for 10 minutes, flow rate 1 mL/minutes, k=254 nm, volume injected: 10 μL).

The results of stability at pH 7 and pH 1 obtained with the various molecules of Examples 6, 7 and 14 are listed in Tables I and II below.

TABLE I
Stability at pH 7
Purity of the hybrid molecules (in
percentage) as a function of time (hours)

| | Time (h) | | | | | | | |
|---|---|---|---|---|---|---|---|---|
| | 0 | 1 | 2 | 4 | 6 | 8 | 15 | 24 |
| PA 1089 | 100 | 100 | 100 | 100 | 98 | 97 | 92 | 83 |
| (Example 6) | | | | | | | | |
| PA 1088 | 100 | 100 | 100 | 100 | — | — | — | 81 |
| (Example 7) | | | | | | | | |
| PA 1074 | 100 | 100 | 100 | 100 | 100 | 97 | 88 | 87 |
| (Example 14) | | | | | | | | |

It results clearly from the results above that these show an excellent stability of the hybrid molecules obtained at the pH tested.

### Example 19

### Antibacterial Activity of the Hybrid Molecules

The antibacterial activity of the hybrid molecules given in the Examples was evaluated by determination of the minimum inhibitory concentrations (MIC) in μg/mL by micromethod in liquid medium and minimum bactericidal concentrations (MBC) in μg/mL by subculture on an agar medium, on various Gram+ and Gram− bacterial species: Staphylococcus aureus CIP 4.83, vancomycin-resistant Enterococcus faecalis CIP 104 676, Streptococcus pneumoniae DSP (decreased sensitivity to penicillins) CIP 104471 and a clinical isolate, Haemophilus influenzae CIP 102514) (inoculation suspension: 10⁸ bacteria/mL, incubation at 37° C., under 5% CO₂ for S. pneumoniae, H. influenzae, S. aureus, and E. faecalis).

The results of the hybrid molecules according to the invention obtained on the various bacterial species indicated above are listed in Tables III and IV below.

TABLE III
MIC and MBC values in μg/ml of an aminoquinoline-penicillin
hybrid molecule on Staphylococcus aureus CIP 4.83

| | MIC (μg/mL) | MBC (μg/mL) |
|---|---|---|
| PA 1007 (Ex. 1) | 0.012 | 0.49 |
| Penicillin G | 0.008 | 0.06 |

TABLE IV
MIC and MBC values in μg/mL of aminoquinoline-cephalosporin hybrid molecules

| | | | | S. pneumoniae | | |
|---|---|---|---|---|---|---|
| | | S. aureus | S. pneumoniae | DSP clinical | E. faecalis | H. inflenzae |
| | | CIP 4.83 | DSP CIP 104471 | isolate | CIP 104 676 | CIP 102514 |
| PA 1046 | MIC | 0.20 | 0.006 | 0.05 | 6.25 | 3.12 |
| (ex. 5) | MBC | >50 | 0.025 | 0.1 | 50 | 25 |
| PA 1089 | MIC | 0.20 | 0.39 | 0.39 | 6.25 | 3.12 |
| (ex. 6) | MBC | 0.39 | 0.39 | 0.39 | 12.5 | >50 |
| PA 1092 | MIC | 12.5 | 1.56 | 6.25 | >50 | 12.5 |
| (ex. 8) | MBC | >50 | 3.12 | 12.5 | >50 | 50 |
| PA 1037 | MIC | 0.39 | 1.56 | 3.12 | 6.25 | 6.25 |
| (ex. 9) | MBC | >50 | 3.12 | 6.25 | 25 | 25 |
| PA 1053 | MIC | 0.78 | 1.56 | 3.12 | 25 | 6.25 |
| (ex. 12) | MBC | >50 | 3.12 | 6.25 | >50 | 25 |
| PA 1074 | MIC | 0.78 | 25 | 25 | 50 | 50 |
| (ex. 14) | MBC | 1.56 | 50 | 50 | >50 | >50 |
| PA 1100 | MIC | 0.20 | 0.20 | 0.39 | 12.5 | 0.78 |
| (ex. 15) | MBC | 25 | 0.78 | 0.39 | 25 | 25 |
| PA 1101 | MIC | 0.20 | 0.20 | 0.20 | 25 | 1.56 |
| (ex. 16) | MBC | 25 | 0.78 | 0.39 | 25 | 12.5 |
| Ceftriax- | MIC | 0.20 | 0.05 | 0.39 | 1.56 | <0.001 |
| one | MBC | >50 | 0.39 | 0.78 | 50 | 1.56 |

It results clearly from Tables III and IV above that the antibacterial activity of the hybrid molecules according to the invention is very significant and perfectly unexpected for the person skilled in the art.

The very high activity is to be noted of these cephaloquine type hybrid molecules on the resistant strains of Stretrococcus pneumoniae. In particular, the molecule PA 1046 has an MIC value equal to 0.006 μg/mL on the strain CIP 104471 and to 0.05 μg/mL on the clinical isolate. As a comparison, ceftriaxone, a third generation cephalosporin, has an MIC of 0.05 μg/mL and 0.39 μg/mL, respectively, on the same strains.

What is claimed is:

1. A compound of the formula (I):
Q-(Y₁)ₚ—(U)_{p′}—(Y₂)_{p″}-A (I)
wherein
Q is an aminoquinoline-type molecule;
A is selected from the group consisting of an antibiotic residue, a derivative of an antibiotic residue, a precursor of an antibiotic residue, a salt of an antibiotic residue, an hydrate of an antibiotic residue, a prodrug of an antibiotic residue, a prodrug salt of an antibiotic residue, a resistance enzyme inhibitor, a resistance enzyme inhibitor salt, and a resistance enzyme inhibitor hydrate, and A and Q are linked together by a covalent bond represented by —(Y₁)ₚ—(U)_{p′}—(Y₂)_{p″}—, the covalent bond being direct or indirect by the use of a spacer arm.

2. A compound of the formula (I):
Q-(Y₁)ₚ—(U)_{p′}—(Y₂)_{p″}-A (I)
wherein
Q is an aminoquinoline of the following formula (II) or (III): 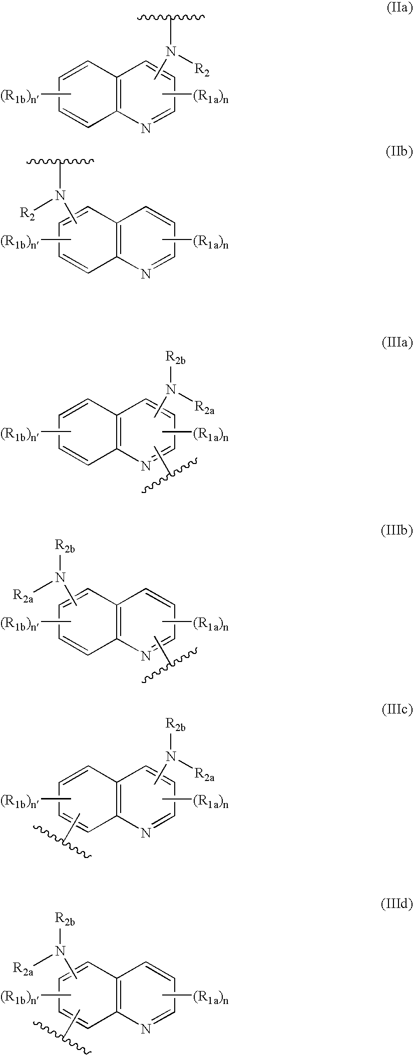
with
n and n′ being, independently of each other, selected from the consisting of 0, 1, 2 and 3,
R₁ₐ and R_{1b} being one or more substituents, identical or different, occupying any position and representing moieties selected from the group consisting of halogen, hydroxy, trifluoromethyl, trifluoromethoxy, carboxy, amine, sulfate, sulfonate, phosphate, phosphonate, nitro, cyano, aryl, heteroaryl, heteroalkyl, alkylamino, alkoxy, alkylthio, alkylsulfonyl, alkylsulfonamido, alkylsulfonylamino, alkylamido, alkylcarboxy, alkoxycarbonyl, and alkylcarbonylamino, said alkyl group comprising 1 to 6 carbon atoms, saturated or unsaturated, linear, branched or cyclic, and optionally containing one or more moieties selected from the group consisting of amine, amide, thioamide, sulfonyl, sulfonamide, carboxy, thiocarboxy, carbonyl, thiocarbonyl, hydroxyimine, ether and thioether moieties, said moieties optionally bearing 1 to 4 substituents, identical or different, selected from the group consisting of halogen, hydroxy, trifluoromethyl, trifluoromethoxy, carboxy, carbonyl, amine, nitro, aryl, and heteroaryl,
R₂ₐ and R_{2b} being substituents, identical or different, optionally forming a cyclic structure together or with Y₁, Y₂, U or A and being selected from the group consisting of a hydrogen atom, and a linear, branched or cyclic C1 to C6 alkyl moiety optionally containing one or more moieties selected from the group consisting of amine, amide, thioamide, sulfonyl, sulfonamide, carboxy, thiocarboxy, carbonyl, thiocarbonyl, ether and thioether moieties, said moieties optionally bearing 1 to 4 substituents, identical or different, selected from the group consisting of halogen, hydroxy, trifluoromethyl, trifluoromethoxy, carboxy, carbonyl, amine, nitro, aryl, and heteroaryl,
p, p′, p″ are, independently of each other, 0 or 1,
Y₁ and Y₂, identical or different, are selected from the group consisting of a saturated or unsaturated, linear, branched or cyclic C1 to C6 alkyl chain optionally containing one or more moieties selected from the group consisting of amine, amide, thioamide, sulfonyl, sulfonamide, carboxy, thiocarboxy, carbonyl, thiocarbonyl, hydroxyimine, ether and thioether moieties, said moieties optionally forming a cyclic structure with R₂ including N of the aminoquinoline part Q and/or the U function, the C1 to C6 alkyl chain optionally bearing 1 to 4 substituents, identical or different, selected from the group consisting of halogen, hydroxy, trifluoromethyl, trifluoromethoxy, carboxy, carbonyl, amine, nitro, hydroxyimine, aryl and heteroaryl, said C1 to C6 chain optionally substituted with 1 to 4 moieties selected from the group consisting of alkyl, alkylamino, alkoxy, alkylthio, alkylsulfonyl, alkylsulfonamido, alkylaminosulfonyl, alkylamido, alkylcarboxy, alkoxycarbonyl, alkylaminocarbonyl, and alkoxyimine type, said alkyl group comprising 1 to 6 carbon atoms, linear, branched or cyclic optionally containing one or more moieties selected from the group consisting of amine, amide, thioamide, sulfonyl, sulfonamide, carboxy, thiocarboxy, carbonyl, thiocarbonyl, ether, thioether, aryl and heteroaryl,
U is a function is selected from the consisting of an amine, amide, thioamide, sulfonyl, sulfonamide, carboxy, thiocarboxy, carbonyl, ether, thioether, thiocarbonyl, sulfonate, alkoxyimine (C═N—OR) and alkoxyiminocarbonyl (C(O)—C═N—OR) function with R representing a hydrogen atom or a linear, branched or cyclic C1 to C6 alkyl moiety optionally containing one or more moieties selected from the group consisting of amine, amide, thioamide, sulfonyl, sulfonamide, carboxy, thiocarboxy, carbonyl, thiocarbonyl, ether and thioether moieties,
said aryl moiety being an aromatic ring having 5 to 6 members optionally bearing one or more substituents selected from the group consisting of: halogen, hydroxy, trifluoromethyl, trifluoromethoxy, carboxy, amine, nitro and cyano;
said heteroaryl moiety being an aromatic ring having 5 to 6 members comprising 1 to 4 heteroatoms selected from the group consisting of nitrogen, sulfur and oxygen, said heteroaryl moiety optionally bearing one or more substituents selected from the group consisting of: halogen, hydroxy, trifluoromethyl, trifluoromethoxy, carboxy, amine, nitro and cyano;
A is selected from the group consisting of an antibiotic residue, a derivative of an antibiotic residue, a precursor of an antibiotic residue, a resistance enzyme inhibitor, a salt of an antibiotic residue, an hydrate of an antibiotic residue, a prodrug of an antibiotic residue and a salt of a prodrug of an antibiotic residue.

3. The compound according to claim 1, wherein the group A is selected from the group consisting of an antibiotic residue, a derivative of an antibiotic residue, a precursor of an antibiotic residue, a salt of an antibiotic residue, an hydrate of an antibiotic residue, a prodrug of an antibiotic residue and a salt of a prodrug of an antibiotic, said antibiotic being selected from the group consisting of a β-lactam, a quinolone, an oxazolidinone, a derivative of fosfomycin, a nitro-imidazole, a nitro-furan, a sulfamide, a streptogramin, a synergistin, a lincosamide, a tetracyclin, a derivative of chloramphenicol, a derivative of fusidic acid, a diaminopyrimidine, an aminoside, a macrolide, a polypeptide, a glycopeptide, a rifamycin, a lipodepsipeptide and an inhibitor of β-lactamase.

4. The compound according to claim 1, wherein the A is selected from the group consisting of:
a β-lactam selected from the consisting of: a penam or penicillin of formula (IV), an oxapenam of formula (V), a penem of formula (VIa) or (VIb), a carbapenem of formula (VIIa) or (VIIb), a cephem or a cephalosporin of formula (VIIIa) or (VIIIb), a cephamycin of formula (IXa) or (IXb), an oxacephem of formula (Xa) or (Xb), a carbacephem of formula (XIa) or (XIb) and a monobactam of formula (XII), as follows 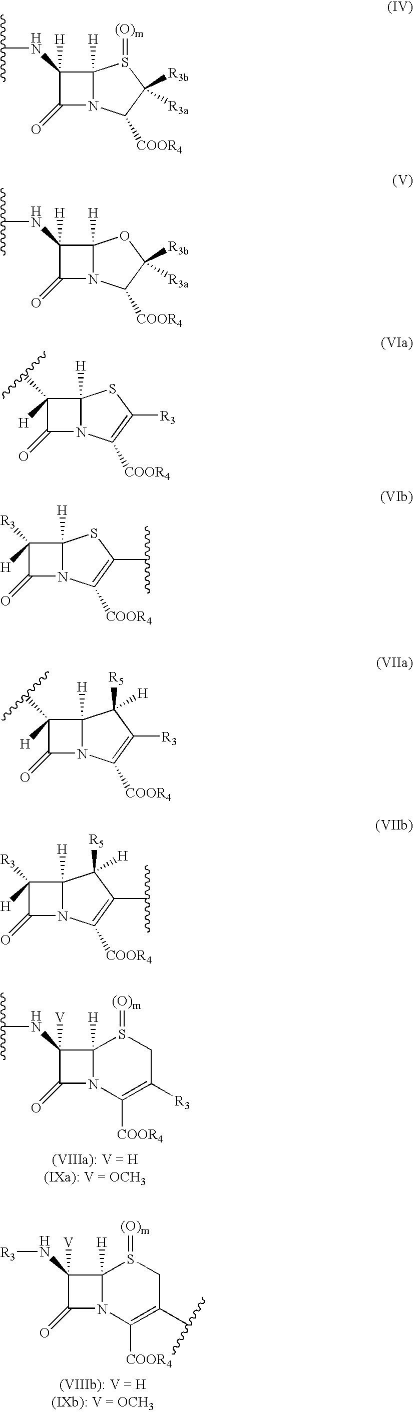 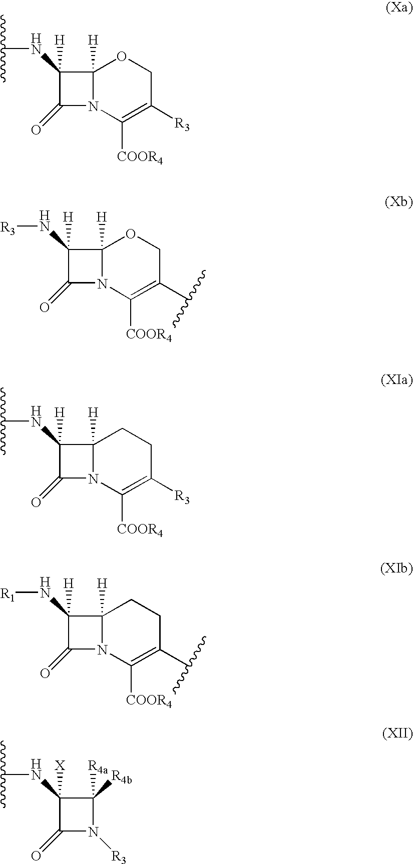
wherein
R₃ₐ and R_{3b} are substituents, identical or different, selected from the group consisting of halogen, hydroxy, trifluoromethyl, trifluoromethoxy, carboxy, amine, sulfate, sulfonate, phosphate, phosphonate, nitro, cyano, aryl, heteroaryl, heteroalkyl, alkylamino, alkoxy, alkylthio, alkylsulfonyl, alkylsulfonamido, alkylsulfonylamino, alkylamido, alkylcarboxy, alkyloxycarbonyl, and alkylcarbonylamino, said alkyl groups comprising 1 to 6 carbon atoms, saturated or unsaturated, linear, branched or cyclic, optionally containing one or more group selected from the group consisting of amine, amide, thioamide, sulfonyl, sulfonamide, carboxy, thiocarboxy, carbonyl, thiocarbonyl, hydroxyimine, ether and thioether moieties, and optionally bearing 1 to 4 substituents, identical or different, selected from the group consisting of halogen, hydroxy, trifluoromethyl, trifluoromethoxy, carboxy, carbonyl, amine, nitro, aryl, and heteroaryl,
R₄ₐ and R_{4b}, identical or different, optionally forming together a cyclic structure or a multiple bond, are a hydrogen atom or a, saturated or unsaturated, linear, branched or cyclic C1 to C6 alkyl moiety, optionally containing one or more moieties selected from the group consisting of amine, amide, thioamide, sulfonyl, sulfonamide, carboxy, thiocarboxy, carbonyl, thiocarbonyl, hydroxyimine, ether and thioether moieties, optionally bearing 1 to 4 substituents, identical or different, selected from the consisting of halogen, hydroxy, trifluoromethyl, trifluoromethoxy, carboxy, carbonyl, amine, nitro, aryl, and heteroaryl,
R₅ is a hydrogen atom or a, saturated or unsaturated, linear, branched or cyclic C1 to C6 alkyl moiety,
V is a methoxy group or a hydrogen atom;
a quinolone of the following formula (XIII), 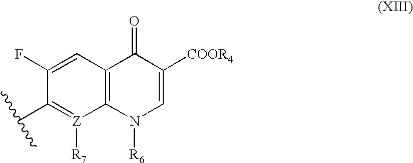
wherein
R₄ is as defined above,
R₆ and R₇ are substituents, identical or different, optionally forming together a cyclic structure or a multiple bond and representing a hydrogen atom or a substituent selected from the group consisting of halogen, heterocycle, aryl, heteroaryl, alkyl, alkoxy and alkylamine moiety, said alkyl groups comprising 1 to 6 carbon atoms, saturated or unsaturated, linear, branched or cyclic, optionally containing one or more moieties selected from the group consisting of amine, amide, thioamide, sulfonyl, sulfonamide, carboxy, thiocarboxy, carbonyl, thiocarbonyl, ether and thioether moieties, optionally bearing 1 to 4 substituents, identical or different, selected from the group consisting of halogen, hydroxy, trifluoromethyl, trifluoromethoxy, carboxy, carbonyl, amine, nitro, aryl, and heteroaryl,
Z is a nitrogen or a carbon atom,
said heterocycle being a 5 to 6 membered-ring comprising 1 to 4 heteroatoms selected from the group consisting of nitrogen, sulfur and oxygen, and optionally bearing one or more substituents selected from the group consisting of: halogen, hydroxy, trifluoromethyl, trifluoromethoxy, carboxy, amine, nitro and cyano;
an oxazolidinone of the following formulae (XIVa) or (XIVb), 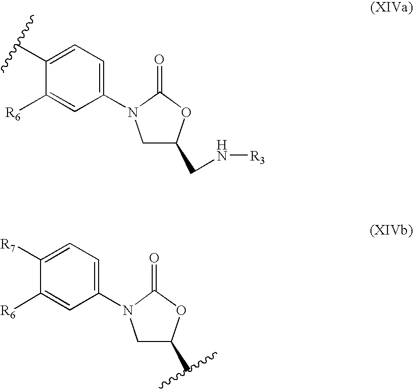
wherein R₃, R₆ and R₇ are as defined above,
a fosfomycin of the following formula (XV), 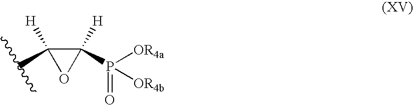
wherein R₄ₐ and R_{4b}, identical or different, optionally forming together a cyclic structure are as defined above;
a nitro-imidazole of the following formula (XVIa), (XVIb), a nitro-furan residue of the following formula (XVII), 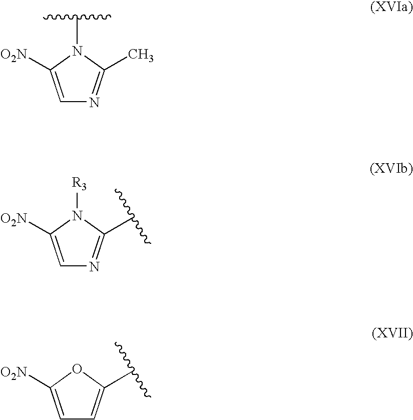
wherein R₃ is as defined above;
a sulfamide of the following formula (XVIII), 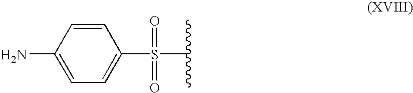
a streptogramin of the following formulae (XIXa), (XIXb), a residue synergistin of the following formula (XX), 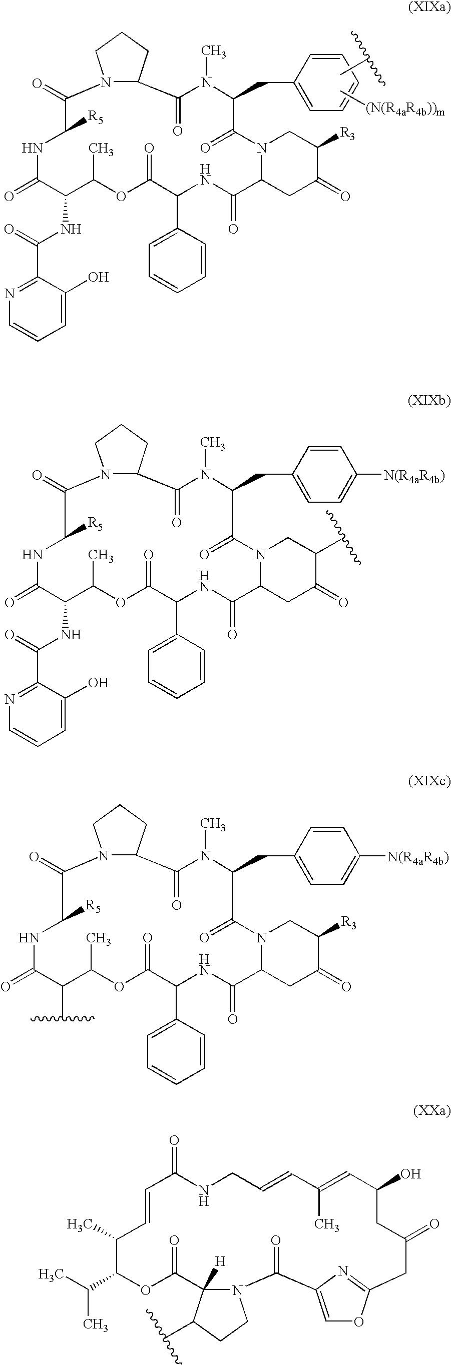 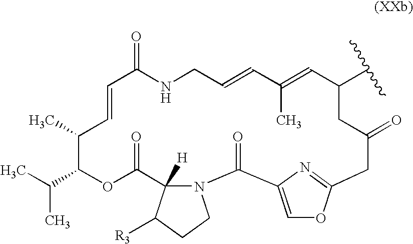
wherein R₃, R₄ₐ, R_{4b}, R₅ and m are as defined above;
a lincosamide of the following formula (XXI), 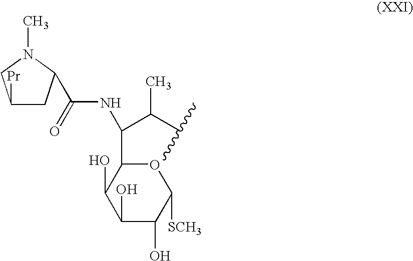
a tetracyclin of the following formula (XXII), 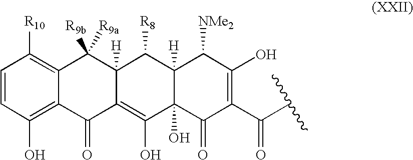
wherein
R₈ and R₉ₐ, R_{9b}, identical or different, are selected from the group consisting of a hydrogen atom, a hydroxy moiety, and a methyl moiety,
R₁₀ is a hydrogen atom or a halogen;
a chloramphenicol of the following formulae (XXIIIa), or (XXIIIb), 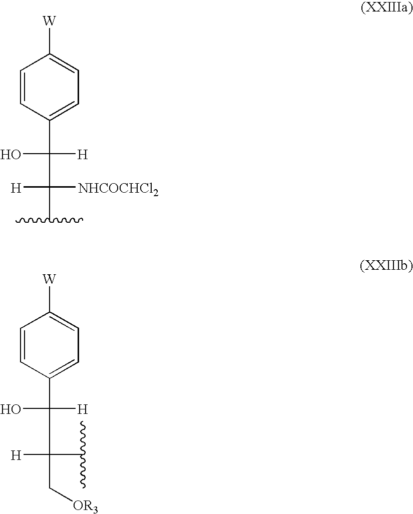
wherein
R₃ is as defined above,
W is an NO₂ or SO₂R₅ moiety, R₅ being as defined above;
a derivative of fusidic acid selected from the group consisting of the following formulae (XXIVa), (XXIVb), and (XXIVc), 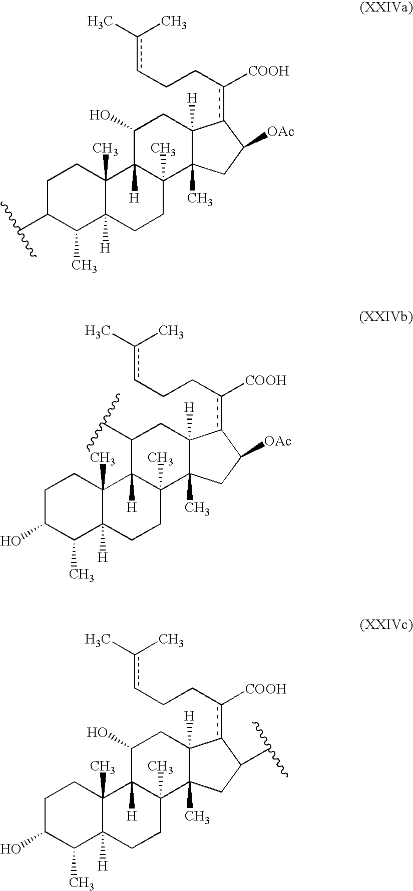
a diaminopyrimidine of the following formula (XXV), 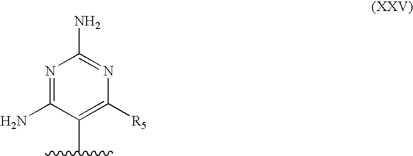
wherein R₅ is as defined above;
an aminoside formed by the union of a genin moiety from the group of aminocyclitols, with one or more oses at least one of which is an aminosugar, linked together via glycosidic bridges;
a macrolide selected from the group consisting of the following formulae (XXVI), (XXVII) and (XXVIII), 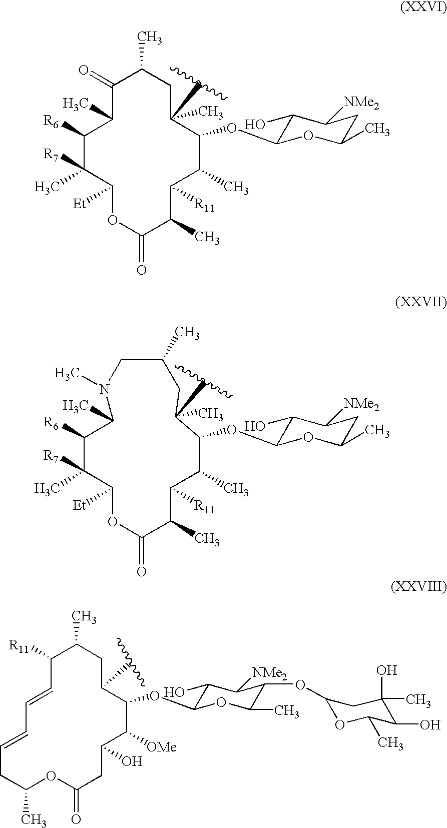
wherein
R₆ and R₇ are as defined above,
R₁₁ is selected from the group consisting of an oxygen atom linked via a double 5 bond of carbonyl type to the macrocycle, a hydroxy group, and an osidic derivative linked via a glycosidic bridge to the macrocycle optionally bearing 1 to 6 substituents, identical or different, selected from the group consisting of hydroxy, alkyl and alkoxy, said alkyl groups comprising 1 to 6 carbon atoms which are linear, branched or cyclic;
a polypeptide selected from the group consisting of a derivative of polymyxines and a derivative of bacitracin linking various peptidic structures;
a glycopeptide selected from the group consisting of a derivative of vancomycin of the formula (XXIX) and derivative of teicoplanin of the formula (XXX), as follows: 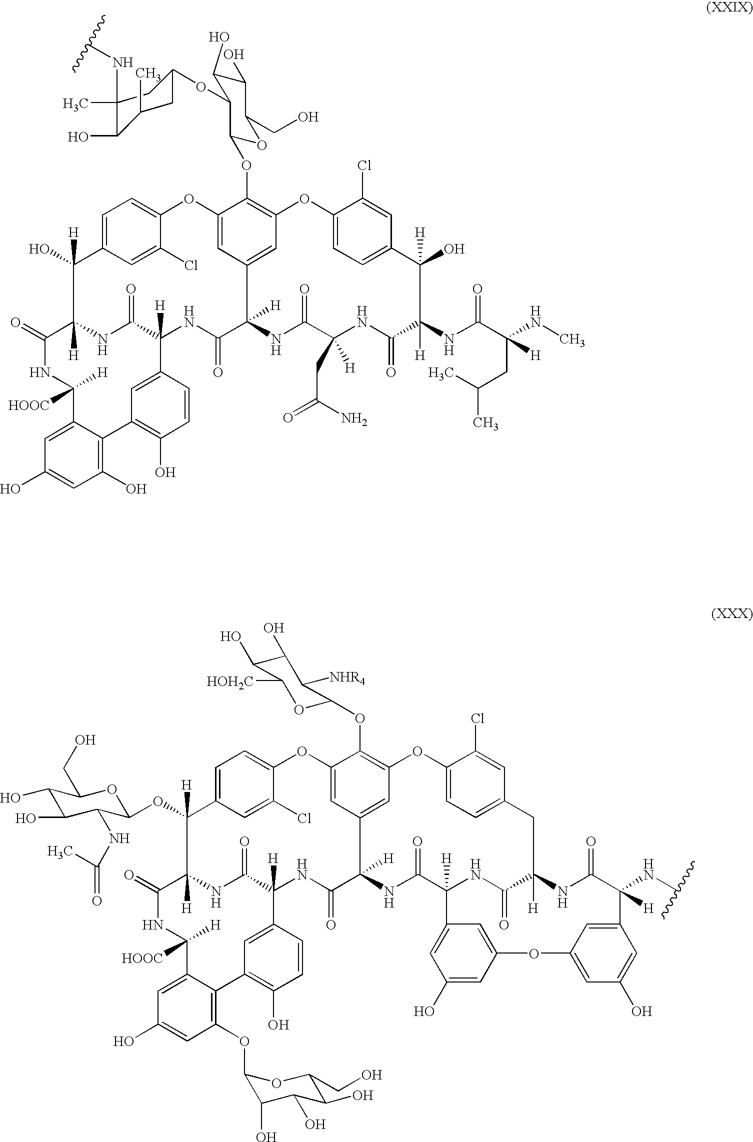
wherein R₄ is as defined above;
a rifamycin of the following formulae (XXXIa) or (XXXIb), 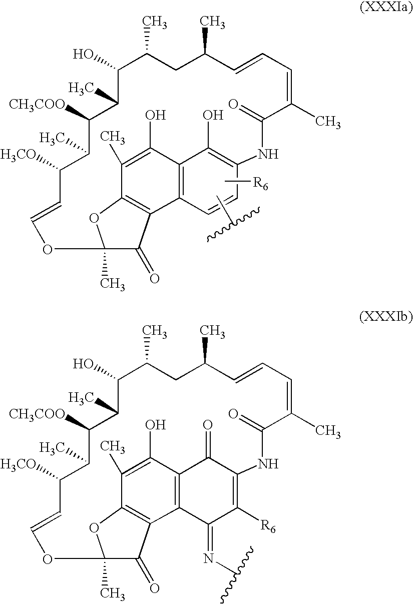
wherein R₆ occupies any position and optionally forming a cyclic structure wherein Y₁, Y₂ or U is as defined above;
a lipodepsipeptide selected from the group consisting of a derivative of daptomycin of the following formula (XXXII), 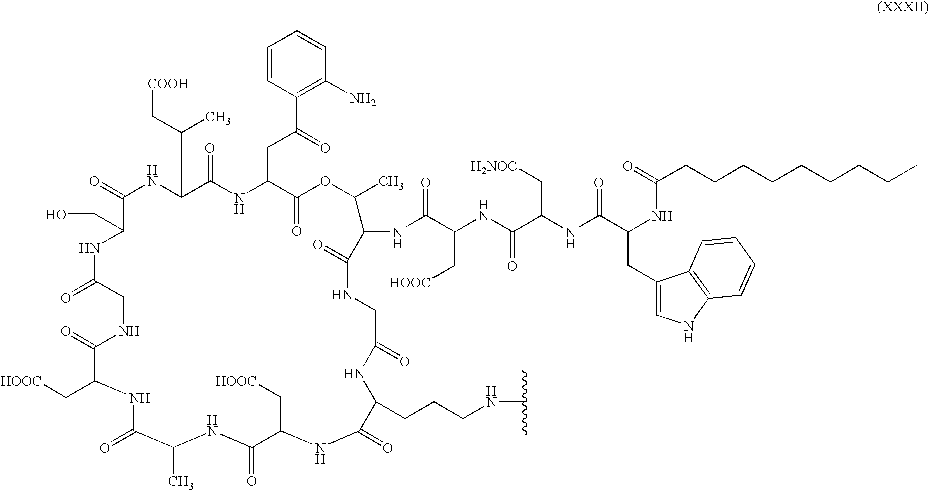
a resistance enzyme inhibitor selected from the group consisting of an inhibitor of β-lactamase of formula (XXXIII), of formula (XXXIV), and of formula (XXXV): 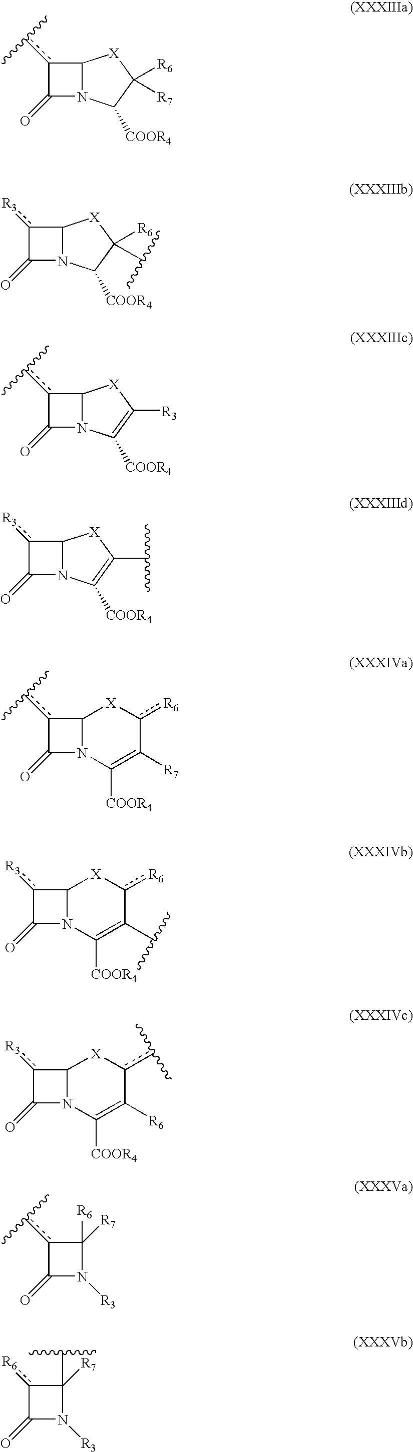 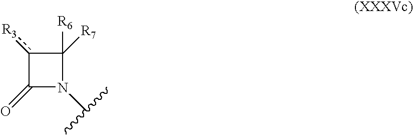
wherein
R₃, R₄, R₆ and R₇ are as defined above,
X is selected from the group consisting of an oxygen atom, sulfur atom, and a group selected from the group consisting of S(O)ₘ, being as defined above, and C(R₅ₐR_{5b}), R₅ₐ and R_{5b}, identical or different, optionally forming together a cyclic structure or a multiple bond, are as defined above;
these compounds having several asymmetric centres are selected from the group consisting of:
a racemic mixture,
an optically pure isomer of compounds of formula (I), their mixtures in any proportions,
an achiral molecule of compounds of formula (I), their mixtures in any proportions,
an acid addition salt, a base addition salt, a zwitterion;
a prodrug of the compounds of formula (I), and a salt thereof.

5. The compound according to claim 1, wherein group Q is a group of formula (IIa) or (IIb) defined according to claim 2.

6. The compound according to claim 1, wherein group A is a group of formula (IV) defined according to claim 4.

7. The compound according to claim 1, wherein group A is a group of formula (VIIIa) or (VIIIb) defined according to claim 4.

8. The compound according to claim 1, wherein group A is a group of formula (XIII) defined according to claim 4.

9. A compound of formula (XXXIX), comprising groups R₁, R₂ and —(Y₁)ₚ—(U)_{p′}—(Y₂)_{p″}— as defined in claim 2 and groups R₄, R₆, R₇ and Z as defined in claim 4,

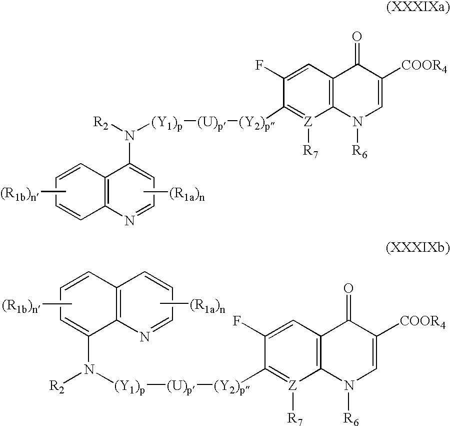

10. The compound according to claim 1, wherein the groups —(Y₁)ₚ—(U)_{p′}—(Y₂)_{p″}— are a group in which p=p′=1 and p″=0, Y₁ and U are as defined in claim 1.

11. The Compound according to claim 10, wherein the groups —(Y₁)ₚ—(U)_{p′}—(Y₂)_{p″}— are selected from the consisting of a methyl, ethyl, propyl, and piperidine group (in including R₂ and N of the aminoquinoline), and U is a carbonyl group.

12. A compound selected from the group consisting of compounds of formula (XXXVIIa), (XXXVIIb), (XXXVIIIa), and (XXXVIIIb), R₁, R₂ and —(Y₁)ₚ—(U)_{p′}—(Y₂)_{p″}— groups being defined according to claim 2, and R₃ and R₄ groups being defined according to claim 4,

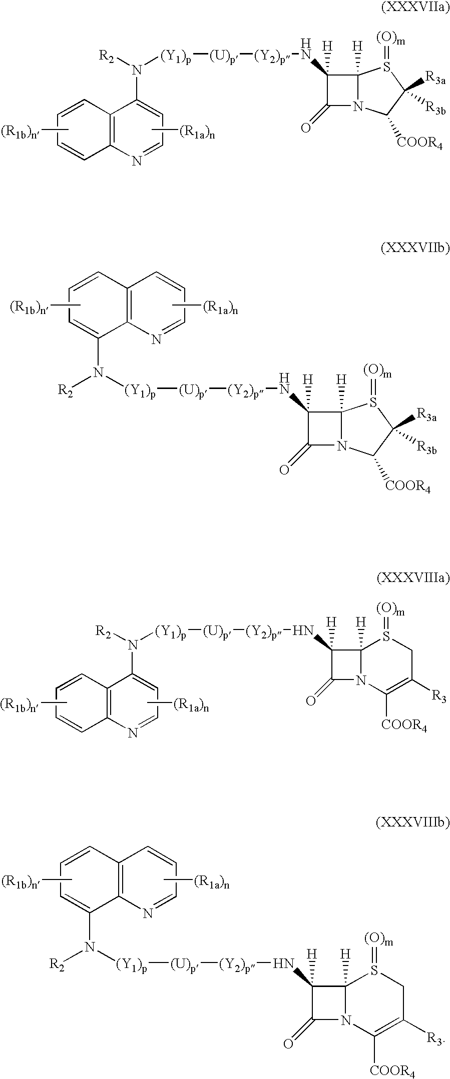

13. The compound according to claim 12, wherein —(Y₁)ₚ—(U)_{p′}—(Y₂)_{p″}— group is a C1 to C6 alkylcarbonyl group,

14. The compound according to claim 12, wherein —(Y₁)ₚ—(U)_{p′}—(Y₂)_{p″}— group is selected from the group consisting of acetyl, propionyl, butyryl, and piperidinecarbonyl (in the case in which R₂ and N are included).

15. The compound according to claim 1, wherein —(Y₁)ₚ—(U)_{p′}—(Y₂)_{p″}— group is a group in which p=p′=0 and p″=1, Y₂ being as defined in claim 2.

16. The compound according to claim 1, wherein —(Y₁)ₚ—(U)_{p′}—(Y₂)_{p″} group is a group in which p=p′=0 and p″=1, Y₂ being a C1 to C6 alkyl chain containing an amine moiety and optionally forming a cyclic structure with R₂ including N of the aminoquinoline.

17. The compound according to claim 15, wherein the —(Y₁)ₚ—(U)_{p′}—(Y₂)_{p″}— group is a piperazine group including N of the aminoquinoline and R₂.

18. An aminoquinoline compound which is coupled via a covalent bond to an antibiotic, wherein the aminoquinoline is selected from the group consisting of:
(2S,5R,6R)-6-{[1-(7-Chloro-quinolin-4-yl)-piperidine-4-carbonyl]-amino}-3,3-dimethyl-7-oxo-4-thia-1-aza-bicyclo[3.2.0]heptane-2-carboxylic acid 2,2-dimethyl-propionyloxymethyl ester;
(2S,5R,6R)-3,3-Dimethyl-7-oxo-6-[3-(quinolin-8-ylamino)-propionylamino]-4-thia-1-aza-bicyclo[3.2.0]heptane-2-carboxylic acid 2,2-dimethyl-propionyloxymethyl ester;
(2S,5R,6R)-6-[2-(7-Chloro-quinolin-4-ylamino)-acetylamino]-3,3-dimethyl-7-oxo-4-thia-1-aza-bicyclo[3.2.0]heptane-2-carboxylic acid 2,2-dimethyl-propionyloxymethyl ester;
(2S,5R,6R)-6-[3-(7-Chloro-quinolin-4-ylamino)-propionylamino]-3,3-dimethyl-7-oxo-4-thia-1-aza-bicyclo[3.2.0]heptane-2-carboxylic acid 2,2-dimethyl-propionyloxymethyl ester;
(6R,7R)-3-Acetoxymethyl-7-[2-(7-chloro-quinolin-4-ylamino)-acetylamino]-8-oxo-5-thia-1-aza-bicyclo[4.2.0]oct-2-ene-2-carboxylic acid;
(6R,7R)-3-Acetoxymethyl-7-[2-(7-chloro-quinolin-4-ylamino)-acetylamino]-8-oxo-5-thia-1-aza-bicyclo[4.2.0]oct-2-ene-2-carboxylic acid hydrochloride;
(6R,7R)-3-Acetoxymethyl-7-[2-(7-chloro-quinolin-4-ylamino)-acetylamino]-5,8-dioxo-5λ⁴-thia-1-aza-bicyclo[4.2.0]oct-2-ene-2-carboxylic acid hydrochloride;
(6R,7R)-3-Acetoxymethyl-7-[2-(7-chloro-quinolin-4-ylamino)-acetylamino]-5,8-dioxo-5λ⁴-thia-1-aza-bicyclo[4.2.0]oct-2-ene-2-carboxylic acid;
(6R,7R)-3-Acetoxymethyl-7-[3-(7-chloro-quinolin-4-ylamino)-propionylamino]-8-oxo-5-thia-1-aza-bicyclo[4.2.0]oct-2-ene-2-carboxylic acid;
(6R,7R)-3-Acetoxymethyl-7-[3-(7-chloro-quinolin-4-ylamino)-propionylamino]-5,8-dioxo-5×4-thia-1-aza-bicyclo[4.2.0]oct-2-ene-2-carboxylic acid;
(6R,7R)-3-Acetoxymethyl-7-[3-(7-chloro-quinolin-4-ylamino)-propionylamino]-5,8-dioxo-524-thia-1-aza-bicyclo[4.2.0]oct-2-ene-2-carboxylic acid hydrochloride;
(6R,7R)-3-Acetoxymethyl-7-[4-(7-chloro-quinolin-4-ylamino)-butyrylamino]-8-oxo-5-thia-1-aza-bicyclo[4.2.0]oct-2-ene-2-carboxylic acid;
(6R,7R)-3-Acetoxymethyl-7-{[1-(7-chloro-quinolin-4-yl)-piperidine-4-carbonyl]-amino}-8-oxo-5-thia-1-aza-bicyclo[4.2.0]oct-2-ene-2-carboxylic acid;
(6R,7R)-3-Acetoxymethyl-7-{[1-(7-chloro-quinolin-4-yl)-piperidine-4-carbonyl]-amino}-8-oxo-5-thia-1-aza-bicyclo[4.2.0]oct-2-ene-2-carboxylic acid hydrochloride;
(6R,7R)-3-Acetoxymethyl-7-[2-(7-trifluoromethyl-quinolin-4-ylamino)-acetylamino]-8-oxo-5-thia-1-aza-bicyclo[4.2.0]oct-2-ene-2-carboxylic acid
(6R,7R)-3-Acetoxymethyl-7-[2-(2-methyl-quinolin-4-ylamino)-acetylamino]-8-oxo-5-thia-1-aza-bicyclo[4.2.0]oct-2-ene-2-carboxylic acid; and
7-[4-(7-Chloro-quinolin-4-yl)-piperazin-1-yl]-1-cyclopropyl-6-fluoro-4-oxo-1.4-dihydro-quinoline-3-carboxylic acid.

19. The compound according to claim 1, wherein A is a cephalosporin.

20. The compound according to claim 1, wherein A is a penicillin.

21. The compound according to claim 1, wherein A is a quinolone.

22. The compound according to claim 1, in the form of a salt selected from the group consisting of an alkali metal salt, a sodium salt, a potassium salt, a lithium salt, an alkaline earth metal salt, a magnesium salt, a calcium salt, an ammonium salt, a salt of nitrogen-containing base, a triethylamine salt, a diisopropylamine salt, an ethanolamine salt, a procainesalt, a N-benzyl-2-phenylethylamine salt, a tris(hydroxymethyl)aminomethane salt, and a N,N′-dibenzylethylnediamine) salt.

23. A method for covalently anchoring a compound Q to compound A via a covalent bond, Q and A being as defined in claim 1.

24. The method according to claim 23, wherein the covalent bond is —(Y₁)ₚ—(U)_{p′}—(Y₂)_{p″}— as defined in claim 2.

25. A method for antibacterial activity comprising use of a compound according to claim 1 as an antibacterial agent.

26. A pharmaceutical composition comprising at least one compound according to claim 1 or 18, in a pharmaceutically acceptable excipient.

27. The pharmaceutical composition according to claim 26, wherein the composition is in a form selected from the group consisting of an injectable, a pulverisable, and an ingestable form, via route selected from the group consisting of intramuscular, intravenous, subcutaneous, intradermal, oral, topical, rectal, vaginal, ophthalmic, nasal, transdermal, and parenteral route.

28. A method for disinfection of medical material comprising using a compound according to claim 1 or 18.

29. The method according to claim 28, for treating medical material contaminated by at least one bacteria selected from the group consisting of Gram+ bacteria, Gram− bacteria, Staphylococcus aureusl Enterococcus faecalis, vancomycin-resistant Enterococcus faecalis, Streptococcus pneumoniae, Streptococcus pneumoniae having dicreased susceptibility to penicillins, and Haemophilus influenzae.

30. A method for treating a bacterial infection of an animal comprising delivering to a subject in need thereof a therapeutically effective amount of a compound according to claim 1 or 18.

31. A method for treating a bacterial infection of a human being comprising delivering to a subject in need thereof a therapeutically effective amount of a compound according to claim 1 or 18.

32. The method according to claim 31, wherein the bacterial infection is due to at least one bacteria selected from the group consisting of Gram+ bacteria, Gram− bacteria, Staphylococcus aureus, Enterococcus faecalis, vancomycin-resistant Enterococcus faecalis, Streptococcus pneumoniae, Streptococcus pneumoniae having dicreased susceptibility to penicillins, and Haemophilus influenzae.

33. A method for treating pneumonia due to strains of Streptococcus pneumoniae which are susceptible or resistant to penicillin, comprising delivering to a subject in need thereof a therapeutically effective amount of compound according to claim 1 or 18.

34. A method for treating meningitis due to strains of Streptococcus pneumoniae which are susceptible or resistant to penicillin, comprising delivering to a subject in need thereof a therapeutically effective amount of compound according to claim 1 or 18.

35. A method for treating otitis due to strains of Streptococcus pneumoniae which are susceptible or resistant to penicillin, comprising delivering to a subject in need thereof a therapeutically effective amount of compound according to claim 1 or 18.

36. A method for treating acute sinusitis due to strains of Streptococcus pneumoniae which are susceptible or resistant to penicillin, comprising delivering to a subject in need thereof a therapeutically effective amount of compound according to claim 1 or 18.

37. A method for treating nosocomial infection due to strains of Staphylococcus aureus, comprising delivering to a subject in need thereof a therapeutically effective amount of compound according to claim 1 or 18.

38. A method for treating infection of the skin or of the skin structure due to strains of Staphylococcus aureus, comprising delivering to a subject in need thereof a therapeutically effective amount of compound according to claim 1 or 18.

39. A method for treating osteomyelitis due to strains of Staphylococcus aureus, comprising delivering to a subject in need thereof a therapeutically effective amount of compound according to claim 1 or 18.

40. An agrifood composition comprising at least one compound according to claim 1 or 18.

41. A method of preparing a compound Q—(Y₁)ₚ—(U)_{p′}—(Y₂)_{p″}-A, as defined in claim 2, comprising anchoring the (Y₁)ₚ—(U)_{p′}—(Y₂)_{p″} group onto an aminoquinoline Q, and then reacting this intermediate compound with A.

42. A method of preparing a compound Q-(Y)ₚ—(U)_{p′}—(Y₂)_{p″}-A, as defined in claim 2, comprising anchoring the (Y₁)ₚ—(U)_{p′}—(Y₂)_{p″} group with A, and then anchoring this intermediate onto an aminoquinoline Q.

43. A method of preparing a compound Q—(Y₁)ₚ—(U)_{p′}—(Y₂)_{p″}-A, as defined in claim 2, comprising anchoring an amino —(Y₁)ₚ—(U)ₚ—(Y₂)_{p″} group onto a corresponding quinoline, enabling an intermediate compound Q—(Y₁)ₚ—(U)_{p′}—(Y₂)_{p″} to be obtained, and then anchoring this intermediate compound onto A.
